SEQUENCE LISTING (50 sequences)

SEQ ID NO: 1 - DNA, 75 bp, synthetic construct
  misc_feature 1..75: note = ARG1A transcript probe
  source 1..75: mol_type = other DNA
acatagagtgggactcttggaatcaggagacaaagctaccacatgtggaaaggtactatgtgtccatgtcattca

SEQ ID NO: 2 - DNA, 85 bp, synthetic construct
  misc_feature 1..85: note = probe sequence ARG1B transcript
  source 1..85: mol_type = other DNA
cttggcttgtttcggacttgctcgggagggtaatcacaagcctattgactaccttaacccacctaagtaaatgtggaaacatccg

SEQ ID NO: 3 - DNA, 88 bp, synthetic construct
  misc_feature 1..88: note = probe sequence for ATG2A transcript
  source 1..88: mol_type = other DNA
gggatgatatccgtgtggttcgatgtattatttttaagctccgtgagtgcgtgggtcagtgtctgcatgaagtggaataaactgccca

SEQ ID NO: 4 - DNA, 138 bp, synthetic construct
  misc_feature 1..138: note = probe sequence BCL2A1 transcript
  source 1..138: mol_type = other DNA
gtagacactgccagaacactattcaaccaagtgatggaaaaggagtttgaagacggcatcattaactggggaagaattgtaaccatatttgcatttgaaggtattctcatcaagaaacttctacgacagcaaattgcc

SEQ ID NO: 5 - DNA, 79 bp, synthetic construct
  misc_feature 1..79: note = BMX transcript probe sequence
  source 1..79: mol_type = other DNA
tgctgctcctgatataacactttccagcctatagcagaagcacattttcagactgcaatatagagactgtgttcatgtg

SEQ ID NO: 6 - DNA, 80 bp, synthetic construct
  misc_feature 1..80: note = CD177 transcript probe sequence
  source 1..80: mol_type = other DNA
cactcattgttatgatgggtacattcatctctcaggaggtgagtgctgcaagcagggccccaaggatgaaggcactggtg

SEQ ID NO: 7 - DNA, 104 bp, synthetic construct
  misc_feature 1..104: note = CEACAM4 transcript probe sequence
  source 1..104: mol_type = other DNA
gagaatggacacggagtttcaggtgagtttctgctaagttcccgtgagcagaaaaagagccaatgagaggaagggtcctctttattcagatcctttcctggtga

SEQ ID NO: 8 - DNA, 78 bp, synthetic construct
  misc_feature 1..78: note = CLEC4D transcript probe sequence
  source 1..78: mol_type = other DNA
ccttccagtccaactgctattttcctcttactgacaacaagacgtgggctgagagtgaaaggaactgttcagggatgg

SEQ ID NO: 9 - DNA, 134 bp, synthetic construct
  misc_feature 1..134: note = CLEC4D_A
  source 1..134: mol_type = other DNA
aaagggcagtcacatccaactttaataaaatatggtggtctttcttaaaattttcaatttgctaatttttcctggatctaagctgaaaaattccaagcaacagctttttaacctaactttcctactactacttt

SEQ ID NO: 10 - DNA, 82 bp, synthetic construct
  misc_feature 1..82: note = HP transcript probe
  source 1..82: mol_type = other DNA
gacaggagtggatgcgataagatgtggtttgaagctgatgggtgccagccctgcattgctgagtcaatcaataaagagcttt

SEQ ID NO: 11 - DNA, 55 bp, synthetic construct
  misc_feature 1..55: note = HPR transcript probe
  source 1..55: mol_type = other DNA
gctgggatcctaagctttgataagagctgtgctgtggctgagtatggtgtgtatg

SEQ ID NO: 12 - DNA, 83 bp, synthetic construct
  misc_feature 1..83: note = IL18R1 transcript probe
  source 1..83: mol_type = other DNA
cactgggagccttcttgatgatctcaaaaataatagctattcaagaaaatcaccaagtgactgtgaaaccgtcagttcggaag

SEQ ID NO: 13 - DNA, 76 bp, synthetic construct
  misc_feature 1..76: note = IL18RAP transcript probe
  source 1..76: mol_type = other DNA
ccctaagatttcccagtggtccgagcagaatcagaaaatacagctacttctgccttatggctagggaactgtcatg

SEQ ID NO: 14 - DNA, 114 bp, synthetic construct
  misc_feature 1..114: note = MMP8 transcript probe
  source 1..114: mol_type = other DNA
gacttcatacatccctcagtttcttaaaatgtcctatgtatatcttctacatgcaatttagaactagattttggttagaagtaaggattataaacaacctagacagtacccttg

SEQ ID NO: 15 - DNA, 81 bp, synthetic construct
  misc_feature 1..81: note = MS4A4A transcript probe
  source 1..81: mol_type = other DNA
acaccacttaatgaggtttgaggccaccaaaagatcaacagacaaatgctccagaaatctatgctgactgtgacacaagag

SEQ ID NO: 16 - DNA, 103 bp, synthetic construct
  misc_feature 1..103: note = PADI4 transcript probe
  source 1..103: mol_type = other DNA
cagccactttcccagtgattagaggcacacagaggctcagggtctcaggatgcgctggaagacagagacacagaagcaagggcagaagcaaagactgggagag

SEQ ID NO: 17 - DNA, 84 bp, synthetic construct
  misc_feature 1..84: note = PFKB2 transcript probe sequence
  source 1..84: mol_type = other DNA
ggttcagcatccagcaactaggagctgcttttctacgtacaaagctgccttcaggaaggctccttaccctgtagcagatgattt

SEQ ID NO: 18 - DNA, 92 bp, synthetic construct
  misc_feature 1..92: note = PLAC8_A transcript probe
  source 1..92: mol_type = other DNA
catcttctcttaggctcctctaaactgtgactgtttctcagcctttccttgtttttgatgaccctgacagttttcaggaggagtagtcaggt

SEQ ID NO: 19 - DNA, 75 bp, synthetic construct
  misc_feature 1..75: note = PLAC8_A transcript probe sequence
  source 1..75: mol_type = other DNA
ggaagccaggtgcctttaatccactgtaacctcacaactccaagtccacagaatatttcaaactgcaggtatgcg

SEQ ID NO: 20 - DNA, 75 bp, synthetic construct
  misc_feature 1..75: note = SIGLEC5 transcript probe
  source 1..75: mol_type = other DNA
ccctcccttggaagaacaaaaggagctccattatgcctcccttagtttttctgagatgaagtcgagggagcctaa

SEQ ID NO: 21 - DNA, 85 bp, synthetic construct
  misc_feature 1..85: note = STOM transcript probe
  source 1..85: mol_type = other DNA
ctggcgggtgacatttgtaacatttcctctttgagactctgagttcacctagagaagtctaagcataacagctttctttcccagc

SEQ ID NO: 22 - DNA, 99 bp, synthetic construct
  misc_feature 1..99: note = TDRD9 transcript probe
  source 1..99: mol_type = other DNA
gactgactttcctctgtgtctgggtgttacagtctgtgcccactgcatcctaaaggccttttctttcttcttttctctttgggtgatagtcagagagtg

SEQ ID NO: 23 - DNA, 93 bp, synthetic construct
  misc_feature 1..93: note = VNN1 transcript probe
  source 1..93: mol_type = other DNA
ttgggtgacattaactgacatttgctttttttcaagacctaatagaaaataagaaagcccataatgtatttagaaacaggaatcctcagagca

SEQ ID NO: 24 - DNA, 93 bp, synthetic construct; identical to SEQ ID NO: 23
  misc_feature 1..93: note = VNN1_2 transcript probe
  source 1..93: mol_type = other DNA

SEQ ID NO: 25 - DNA, 75 bp, synthetic construct
  misc_feature 1..75: note = ZDHHC20 transcript probe
  source 1..75: mol_type = other DNA
gggattcacagaagcactactccagagcagaatgatgccttaatcttaagtgtccatttgtgcagcattgactta

SEQ ID NO: 26 - DNA, 110 bp, synthetic construct
  misc_feature 1..110: note = ARG1A transcript
  source 1..110: mol_type = other DNA
aaataagcacacttacataagcccccatacatagagtgggactcttggaatcaggagacaaagctaccacatgtggaaaggtactatgtgtccatgtcattcaaaaaatg

SEQ ID NO: 27 - DNA, 110 bp, synthetic construct
  misc_feature 1..110: note = ARG1B transcript
  source 1..110: mol_type = other DNA
agcagttgcaataaccttggcttgtttcggacttgctcgggagggtaatcacaagcctattgactaccttaacccacctaagtaaatgtggaaacatccgatataaatct

SEQ ID NO: 28 - DNA, 110 bp, synthetic construct
  misc_feature 1..110: note = ATG2A transcript
  source 1..110: mol_type = other DNA
gtgggcagtttattccacttcatgcagacactgacccacgcactcacggagcttaaaaataatacatcgaaccacacggatatcatcccctcctccccccagacacagaa

SEQ ID NO: 29 - DNA, 110 bp, synthetic construct
  misc_feature 1..110: note = BCL2A1 transcript
  source 1..110: mol_type = other DNA
ctccttataggtatccacatccggggcaatttgctgtcgtagaagtttcttgatgagaataccttcaaatgcaaatatggttacaattcttccccagttaatgatgccgt

SEQ ID NO: 30 - DNA, 110 bp, synthetic construct
  misc_feature 1..110: note = BMX transcript
  source 1..110: mol_type = other DNA
atttagatcaaattagtaattttgtttatgctgctcctgatataacactttccagcctatagcagaagcacattttcagactgcaatatagagactgtgttcatgtgtaa

SEQ ID NO: 31 - DNA, 618 bp, synthetic construct
  misc_feature 1..618: note = CD177 transcript
  source 1..618: mol_type = other DNA
atgacctgccccaggggcgccactcattgttatgatgggtacattcatctctcaggaggtgagtgctgcaagcagggccccaaggatgaaggcactggtggcctggactcctgggtctgagggaggaggggctgggggcctggactcctggtccgagggaggaggcgctgggggcctggactcctggtccgagggaggaggggctgggggcctggactcctggtctgagggaggaggcgctgggggcctgggctcctggtcccagggaggaggggctgggtgcctggactcctggtctgagggaggaggggctgggggccggggctcctgggtctgaggagctgaggctctggactcctgggtctgaggtaggagggactgggggcctggactcctgggtctgagggaggaggggctgggggcctggacccctgggtctgaggagctggagctgggggtctgggctcctcggtctgagggaggagggtctggggcctggactcctgggtttacaacttggctgggctgtactctgtgtcctttctgacttggtcttctccctctaggtgggctgtccaccaaaatgagcattcagggctgcgtggcccaaccttccag

SEQ ID NO: 32 - DNA, 110 bp, synthetic construct
  misc_feature 1..110: note = CEACAM4 transcript
  source 1..110: mol_type = other DNA
gagaatggacacggagtttcaggtgagtttctgctaagttcccgtgagcagaaaaagagccaatgagaggaagggtcctctttattcagatcctttcctggtgaccccgg

SEQ ID NO: 33 - DNA, 110 bp, synthetic construct
  misc_feature 1..110: note = CLEC4D transcript
  source 1..110: mol_type = other DNA
ctggagagccttccagtccaactgctattttcctcttactgacaacaagacgtgggctgagagtgaaaggaactgttcagggatgggggcccatctgatgaccatcagca

SEQ ID NO: 34 - DNA, 110 bp, synthetic construct
  misc_feature 1..110: note = CLEC4D_A transcript
  source 1..110: mol_type = other DNA
caaccagaaattatgcttttctggtgcatgaaacattaattgcaaagggcagtcacatccaactttaataaaatatggtggtctttcttaaaattttcaatttgctaatt

SEQ ID NO: 35 - DNA, 110 bp, synthetic construct
  misc_feature 1..110: note = HP transcript
  source 1..110: mol_type = other DNA
gacgggagtggacaggagtggatgcgataagatgtggtttgaagctgatgggtgccagccctgcattgctgagtcaatcaataaagagctttcttttgacccatttctgt

SEQ ID NO: 36 - DNA, 110 bp, synthetic construct
  misc_feature 1..110: note = IL18R1 transcript
  source 1..110: mol_type = other DNA
acacctggtacgcggctgggatcctaagctttgataagagctgtgctgtggctgagtatggtgtgtatgtgaaggtgacttccatccagcactgggttcagaagaccata

SEQ ID NO: 37 - DNA, 110 bp, synthetic construct
  misc_feature 1..110: note = IL18R1 transcript
  source 1..110: mol_type = other DNA
taccccccaaagggagcccagcactgggagccttcttgatgatctcaaaaataatagctattcaagaaaatcaccaagtgactgtgaaaccgtcagttcggaaggctggt

SEQ ID NO: 38 - DNA, 110 bp, synthetic construct
  misc_feature 1..110: note = IL18RAP transcript
  source 1..110: mol_type = other DNA
tgggtactttcagtacacaacacccctaagatttcccagtggtccgagcagaatcagaaaatacagctacttctgccttatggctagggaactgtcatgtctaccatgta

SEQ ID NO: 39 - DNA, 110 bp, synthetic construct
  misc_feature 1..110: note = MMP8 transcript
  source 1..110: mol_type = other DNA
gatatacataggacattttaagaaactgagggatgtatgaagtcagataggcaagtaatataacaataaatcctagaagtcagatatgtaagtattgaaatagtaaatat

SEQ ID NO: 40 - DNA, 110 bp, synthetic construct
  misc_feature 1..110: note = MS4A4A transcript
  source 1..110: mol_type = other DNA
cacaccacttaatgaggtttgaggccaccaaaagatcaacagacaaatgctccagaaatctatgctgactgtgacacaagagcctcacatgagaaattaccagtatccaa

SEQ ID NO: 41 - DNA, 481 bp, synthetic construct
  misc_feature 1..481: note = PADI4 transcript
  source 1..481: mol_type = other DNA
gacagaaaaaaaacagcagaaaataaagaacattctgtcaaacaagacattgagagaacataattcatttgtggaggtaggagcctgggtgcctacaccccagcagacctgacgccctgtccccggctcagccactttcccagtgattagaggcacacagaggctcagggtctcaggatgcgctggaagacagagacacagaagcaagggcagaagcaaagactgggagaggctgagggagcagagggaatgggaggccccagggtcccccgagagcactggccagaggcccctctgtgcagtgaggcctggcagccaccttcactgccttcctgacactgtcccaggtcctaccctccggcagggggcctcagccccacactgtcccccacccccacccccgactgccatcagtcccccactcactgcccctgccccttccccaagagatgcatcgactggaaccgcgagctgctgaagc

SEQ ID NO: 42 - DNA, 110 bp, synthetic construct
  misc_feature 1..110: note = PFKFB2 transcript
  source 1..110: mol_type = other DNA
gaaaagtaatgatatgtagcttttctcaaatggttcttttatactgtggatgatacaggactctgttacctaagatgtgataagctgggctgcaggcggttcagcatcca

SEQ ID NO: 43 - DNA, 3816 bp, synthetic construct
  misc_feature 1..3816: note = PLAC8A transcript
  source 1..3816: mol_type = other DNA
tgcaaaagatggtgaagaactcaagctgaagaggtgtctgctgaattttgttgcttcggtaagagcttttcacctataaaggacacacaagagttttgaattttcttaaggtacttttataatttttaataaacttttaattgcagaatcatttttgattgacggaaaagttgcaaagatagtacagcgagctctcatgtatctggcacccaattttcctgttggttaacatcttacattaccatgggaggtttgtcaaaactaatgaacccatactgatgcattactactaatcaaagcccatactaccttcagattactcagtttttacctactttaattttctgttccagcatctcatccagcataccacattacatttaattgtcatcttctcttaggctcctctaaactgtgactgtttctcagcctttccttgtttttgatgaccctgacagttttcaggaggagtagtcaggtattttgtggaatgtgcctcagtttaggtttgtctgatgtttttctcatggttagactggagttaagaggttttggggagaagaccacaaaggtaaagtgccattctcatcacatcaaatcaggggtgcaaactatcaatatgacttattaccaatgatggtgacgttgattacctggctcaggtcattgtgtcaggtttctccattgtggagttactccatacagtatagagagtgcacttgcatgctgtacttttcatgctgtactttttggaaggaagtcactatgcaaacccccacttcagggtggggaatcatgccgctctccttgaggaaggagtatctacctaaatatttggagttccatatgggagatgtgtctcttctcccatacagaaattctcctgccacttctttgctctcaaaattcccagttttccctgctttcaacatacatactttttttcaattttcaaattttattcagtcatttatgtcagcacaaactcatagatattttatactttgggttataatctaacacgaccttattttgttcctcaaagtgccccagtttggactatggggagcttttccagtggattcctatgtccttcatctcaatctattttaacgtaaatcatgttgttattccttgctctttctctcttgtttagctccccatttcgatatctgtttgtctaaagttttattgccagtgagtgatgtagggtccaagttgatgaggaggagtcaggaccgccaaggtattttacttcaaacccacctaaagattcagtgagatattttccctgaggttaaagaaaacacttgagctatcaggactcattactatgaatgtgtgggtagatatttgatccaaattgtttcatgtatttatttaagtaaacagtgttggaatgatctaactccccatctttgcttctggcctcatcctgtacaatctgtactatatactattattagattaatatttattattttaaaattttatttatttttatttttactttttagtagagatgaggtcttgctatgttgcccaggctggtcgctggtgctgaactcctgagctcaagtgatcctcccacctcggtctcccaaagtgttgggattataggcatgagccaccatgcccagccttagattcatatttctaaagagtagttctattcatttacttcaagcctgcaataatttcccaattcctaccaagtcattagcttagtattcagacatcgcatgctaaggcctttaaaatcttttatttttcgttatttttctacttatgccatatgttctagccaagttggacctaagctcataaagatattttctctgtttttgcctctctagtttcattaatattgctgattagaagcaacatttcatttcttttctttttcttttcctttttttttttttttttttttttgagacagggctggagtgcagtggtgtgatcatagctcactgcagcttctacctcccaggctcaagtgatcctcccacctcagcctcccaagtatctgggactacaggcgtgcaccaccacacctagctaatttttgtattttttcaagagacagggtttcaacatgttccccaggctggtcttgaactcctgggttcaagcagtcctcttgccttggcctcccaaagtgctgggatcacaggtgtgagccactgcactcagcctccatttctattaattaaaaccacatcttgagtcagtgatcctctgtttgcccctatctgatttacttactcccttgaccatcctgctgtgtgccctgggaagcgacctgcggactaccgtgacactttgctgccctctgcctttctggtgggttcagccaatgggaaaaccagaaggacatccgaaagtgaggtcagggtactttttcagttctatgggttgcctcgggctgctcacaaccctctcttgagaggatactgtttctttcagggcaggcctcttcacacagctctctccttaaggattcaggtcactgctccctcttctcctccctggggctgttgccaggcaggttattatgccatcccttgtcatctccctacaatgccccacccacacctttataattagtcttcattaaatctttgaactatcacagtttgagtgccatctgtttcttgttgagaactaatacgcacttcccctttagaattaaaaatcactgctttcaagaaactttcacctatacatcttacaagatgtgatcttttcctcctctgaacataaaataccgctttgtctataggcattaattgaattctgcattttagttaggggtttgtctaatccccctttttcgtataaattctataatgacagagggactagcttatttatcttgctccccaaccctactacaatacctaacagtatcttacctagtcttagatattgatacattgatacattagttgctaagataatatatacttgatcctagatccttaatacattacttgctaagtaaatgaacatttttttacgtatctcccaagagttatttttattaacccatacagagtccttatcacttcaggaaggaaactggatattacaaagagtagaaaaattgtgttcttcccatacttaatatatattctacaggggatggagtaagtcatgttgatttctacttaactacatcctcaatagatagtcctgagaaaaattagttgaaaatgacttgattgaatttcagatggttaattctatataacatttccaaacattctaaattctatgttcacctctttcaaagttcagcttctccaacaggagttttctatttaacctcaccttgctatgatagatgctttatgccacaatcaatccaaatatcatacagaatgttattttctttactcttctattttataattatttacgaattccttttgtcaacttgtttgtcttagcagggaacatgttttctaattcttttgtatctcaccatactctggtattttacaagtggtaagtgcttccttaatagatcaccagaattctgagaagtttagaaaattcaccattcctttatttactaaaatatggtttcaaagtatagagctatcttcttcataagaagaaaattaaagccaatcttgatattatgatagcacttcaggagacttaagaaatagttattgctgaattattcaaaacaacaaataattgacaaagtctttagtgatatttctaaaaacccacatgttctgagaggcatgtttgcattgactcaccatcagtttttagaaagtacgcatggctctccttctg

SEQ ID NO: 44 - DNA, 109 bp, synthetic construct
  misc_feature 1..109: note = RNASE2 transcript
  source 1..109: mol_type = other DNA
gcaaaaattgtcaccacagtggaagccaggtgcctttaatccactgtaacctcacaactccaagtccacagaatatttcaaactgcaggtatgcgcagacaccagcaaa

SEQ ID NO: 45 - DNA, 110 bp, synthetic construct
  misc_feature 1..110: note = SIGLEC5 transcript
  source 1..110: mol_type = other DNA
tgcttggggcctcctggtccttaggctccctcgacttcatctcagaaaaactaagggaggcataatggagctccttttgttcttccaagggaggggcatccccaggagga

SEQ ID NO: 46 - DNA, 110 bp, synthetic construct
  misc_feature 1..110: note = STOM transcript
  source 1..110: mol_type = other DNA
ctcgtgctgggaaagaaagctgttatgcttagacttctctaggtgaactcagagtctcaaagaggaaatgttacaaatgtcacccgccagctttctggccagtaagcaga

SEQ ID NO: 47 - DNA, 110 bp, synthetic construct
  misc_feature 1..110: note = TDRD9 transcript
  source 1..110: mol_type = other DNA
ctaaaggccttttctttcttcttttctctttgggtgatagtcagagagtggtgtttttgttcaggtgggaaggattggaaactctagtcttttctagaaacagaaaatca

SEQ ID NO: 48 - DNA, 110 bp, synthetic construct
  misc_feature 1..110: note = VNN1 transcript
  source 1..110: mol_type = other DNA
atgttttctgttttacattgaaattatatgagaatacagagaattgctctgaggattcctgtttctaaatacattatgggctttcttattttctattaggtcttgaaaaa

SEQ ID NO: 49 - DNA, 110 bp, synthetic construct; identical to SEQ ID NO: 48
  misc_feature 1..110: note = VNN1_2 transcript
  source 1..110: mol_type = other DNA

SEQ ID NO: 50 - DNA, 110 bp, synthetic construct
  misc_feature 1..110: note = ZDHHC20 transcript
  source 1..110: mol_type = other DNA
tttccagaataacattaagtcacttttgtagctctaagtcaatgctgcacaaatggacacttaagattaaggcatcattctgctctggagtagtgcttctgtgaatccca

(19) United States
(12) Patent Application Publication
(10) Pub. No.: US 2025/0188541 A1
(43) Pub. Date: Jun. 12, 2025

(54) METHOD OF MANAGING CLINICAL OUTCOMES FROM SPECIFIC BIOMARKERS IN BURN PATIENTS

(71) Applicant: THE UNITED STATES GOVERNMENT, AS REPRESENTED BY THE SECRETARY OF THE ARMY, Fort Detrick, MD (US)

(72) Inventors: Rasha HAMMAMIEH, Germantown, MD (US); Nabarun CHAKRABORTY, Rockville, MD (US); Ruoting YANG, Fredrick, MD (US); Ross CAMPBELL, Baltimore, MD (US); Alexander B. LAWRENCE, Fort Detrick, MD (US); Marti JETT-TILTON, Washington, DC (US); Jeffrey SHUPP, Washington, DC (US); Aarti GAUTAM, North Potomac, MD (US); Melissa MCLAWHORN, Arlington, VA (US)

(21) Appl. No.: 18/688,169

(22) PCT Filed: Aug. 30, 2022
(86) PCT No.: PCT/US2022/075665

§ 371 Date: Feb. 29, 2024

Related U.S. Application Data
(60) Provisional application No. 63/238,364, filed on Aug. 30, 2021.

(51) Int. Cl. C12Q 1/6883; G16H 50/20; G16H 50/70

(52) U.S. Cl. CPC C12Q 1/6883; G16H 50/20; G16H 50/70; C12Q 2600/118; C12Q 2600/158

(57) ABSTRACT

Inventors unexpectedly discovered and claim a method of managing clinical outcomes using clinical outcome burn patient management system and method to curate a panel of optimum number of biomarkers to describe the clinical outcome variable with maximum efficacy for clinicians managing treatment and determining clinical outcomes for burn patients.

### STATEMENT AS TO RIGHTS OR INVENTIONS MADE UNDER FEDERALLY SPONSORED RESEARCH AND DEVELOPMENT

The invention was made with government support from the Bacterial Diseases Branch, Walter Reed Army Institute of Research (WRAIR). The United States Government has certain rights in the invention.

### FIELD

Methods for curating a panel of optimum number of biomarkers to describe the clinical outcome variable with maximum efficacy for clinicians managing treatment and determining clinical outcomes for burn patients.

### BACKGROUND

Sepsis is highly prevalent among the soldiers injured in combat and thermal injury is widespread within the context of the War. Thanks to the great accomplishments of combat causality care, 95% of burn patients survive, however burn patients are most vulnerable to sepsis¹⁷. Once sepsis is suspected or diagnosed it must be treated expeditiously. Every hour that a patient with sepsis does not receive treatment, an 8% increase in mortality is observed^{17,18}.

Sepsis is a life-threatening condition with increasing incidence (17% increase between 2000-2010)⁶ that is generally attributed to a bacterial infection or, less frequently, from a fungal or viral infection. Incidents of sepsis are highly widespread among hospitalized patients, accounting for nearly 1 out of every 23 hospitalized patients⁶⁻¹⁰. Sepsis is a leading healthcare burden, with an aggregate cost of $15.4 billion in 2009^{6,10}, whereas nonspecific diagnoses of sepsis account for another $23.7 billion each year^{11,12}. The growing incidence of sepsis, most disturbingly is accompanied by high mortality that have surged 31% between 1999 and 2014¹³. It has been estimated that approximately 30,000 sepsis-related deaths occur annually, with particularly high rates in critically ill patients admitted to intensive care units (ICUs)^{6,14,15}

In 2016, a task force consisting of experts in sepsis pathobiology, clinical trials, and epidemiology was convened by the Society of Critical Care Medicine and the European Society of Intensive Care Medicine. They recognized that sepsis is a syndrome without, at present, a validated criterion standard diagnostic test¹⁶.

Systemic Inflammatory Response Syndrome (SIRS) and quick sequential organ failure assessment (qSOFA) based diagnosis has been criticized for their delayed detection because the clinical signs of sepsis need to have been present¹⁷. FDA approved tools to ID pathogens demonstrated high false positive readings; and its reason is discussed in the following section.

The performance of SeptiCyte Lab was optimized using post-surgical critically-ill patients as documented in clinicaltrials.gov (NCT02127502) and reported elsewhere^{4,18}. Since, this cohort has less concentration of burn patients and burn patients' sepsis pathophysiology is very different from that of the critically ill patients⁵, we subscribe an urgent need for burn patients specific sepsis markers.

In a review named “Sepsis in the burn patient: a different problem than sepsis in the general population”⁵, DG Greenhalgh mentioned that “there are several differences between sepsis in the general population and sepsis found after a burn injury”. Burn patients lose the first barrier to infection—their skin. The burn patient is continuously exposed to inflammatory mediators as long as the wound remains open. When there are extensive burns the exposure to pathogens will persist for months. Therefore, all burns >15-20% TBSA will have a persistent “SIRS” that persists for months after the wound is closed.” Furthermore, the diagnosis of sepsis in patients with severe burns (>20% of TBSA) is particularly complicated by the overlap of clinical signs of the post-burn hypermetabolic response with those of sepsis¹⁹.

Procalcitonin (PCT) has been promoted as the burn sepsis markers by certain perspective studies²⁰, however independent studies reported suboptimal performances of PCT^{17,21}. At baseline burn patients persist in a hyper-inflammatory state. This inflammatory state has features that are consistent with sepsis (tachycardia, leukocytosis, febrile episodes and derangements in end-organ perfusion for burn shock). Hence, there is a critical gap in finding markers for burn sepsis⁵.

### SUMMARY

A method for managing clinical outcomes for a mammalian subject suffering burns, said method comprising the steps of: (a) obtaining biomarker data from the burn subject and comparing the biomarker data from the burn subject to corresponding biomarker data from transcriptomic clinical studies for a comparative group of burn subjects further comprising a spectrum of increasing severity of biomarkers for all burn subjects, Early vs. Late cohorts, wherein the biomarker data is segregated to a (1) training set of biomarker data and (2) a test set of biomarker data, producing a prediction of clinical outcomes for the burn subject by selecting high performing features by a logistic regression data shape model fitting algorithm; (b) logistic regression algorithm and assigning unique weighing factors to each of the selected features to make a best fitting model that would distinguish Early vs. Late cohorts; and (c) obtaining a clinical outcome priority flow chart and/or list for the burn subject by estimating the area under the curve (AUC) values of the receiver operating characteristic (ROC) curve.

Another embodiment pertains to an apparatus that includes a polymerase chain reaction (PCR) device configured to measure first data that indicates biomarker values for one or more biomarkers collected from a sample of a burn subject; and at least one processor connected to the PCR device to receive the first data of the one or more biomarker values; and at least one memory including one or more sequence of instructions. The at least one memory and the one or more sequence of instructions are configured to, with the at least one processor, cause the apparatus to perform at least the following;
apply coefficients to the values for the one or more biomarkers, and
determine second data that indicates a prediction that the burn subject will develop sepsis based on applying the coefficients to the biomarker values for the one or more biomarkers.

### BRIEF DESCRIPTION OF THE DRAWINGS

### Figure Legends

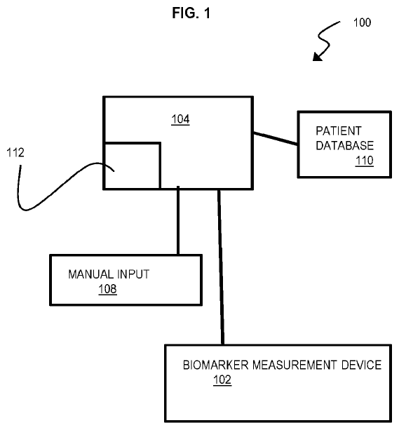

FIG. 1 is a block diagram that illustrates an example of an apparatus for predicting that a burn patient will develop sepsis, according to one embodiment.

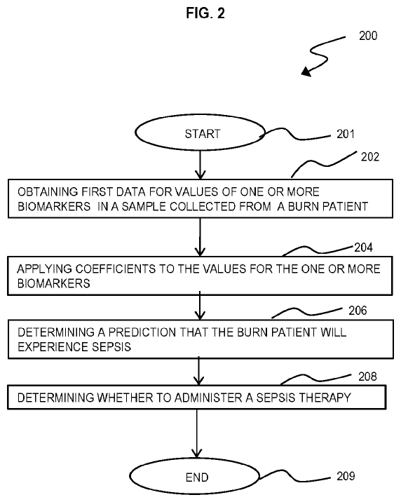

FIG. 2 is a flow diagram that illustrates an example of a method for predicting whether burn patient will develop sepsis, according to one embodiment.

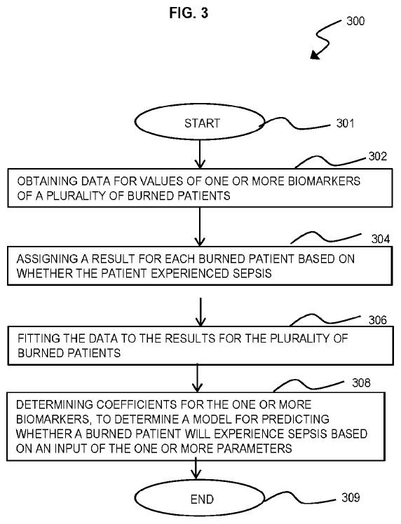

FIG. 3 is a flow diagram that illustrates an example of a method for determining a model for predicting whether a burn patient will develop sepsis, according to one embodiment.

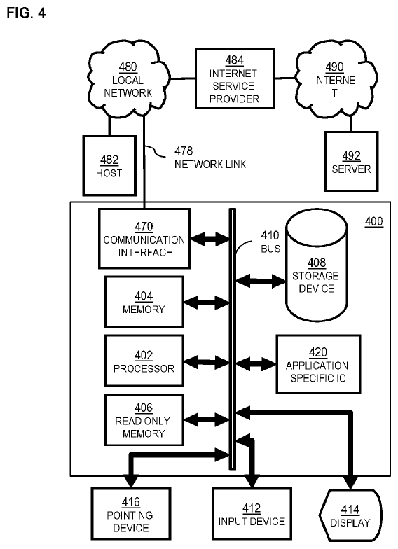

FIG. 4 is a block diagram that illustrates a computer system upon which an embodiment of the invention may be implemented.

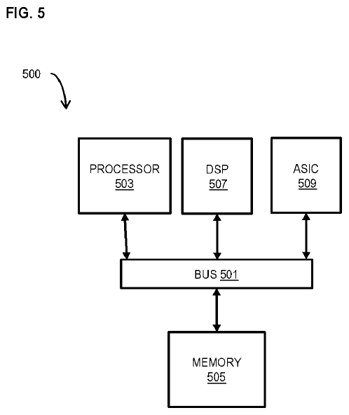

FIG. 5 is a block diagram that illustrates a chip set upon which an embodiment of the invention may be implemented.

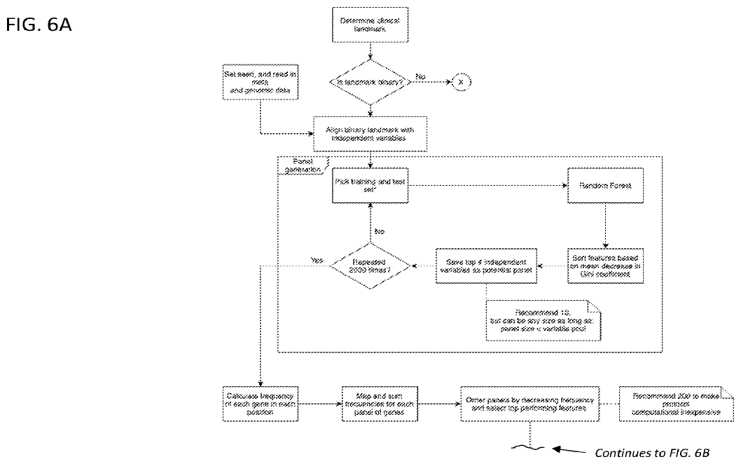

FIG. 6. Flow chart shows the decision tree towards finding the robust biomarker panel to define the binary outcome variable. The Flow chart is broken into two Figures, FIG. 6A and FIG. 6B which are connected by the wavy line as indicated.

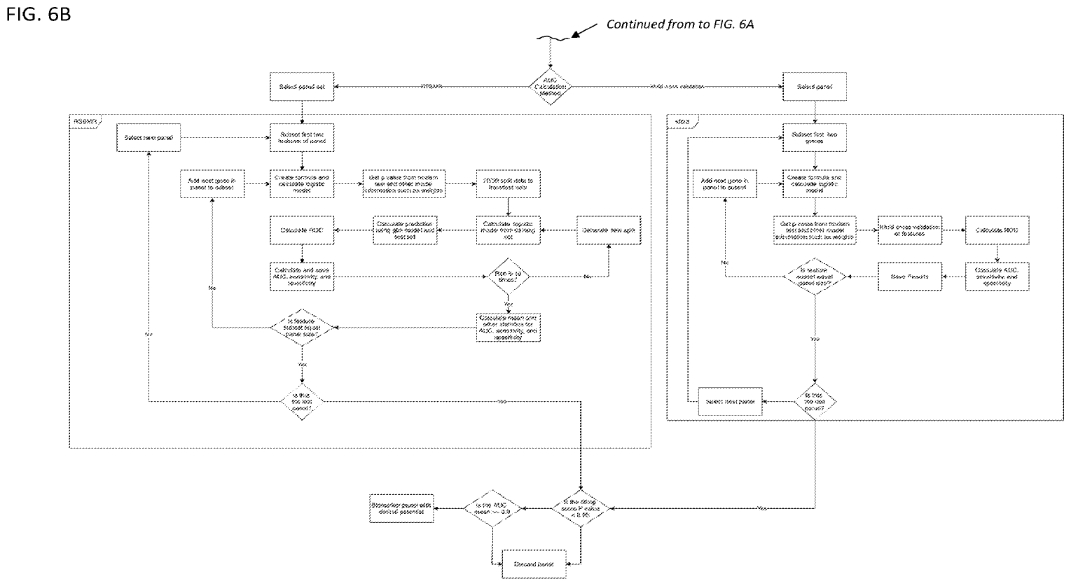

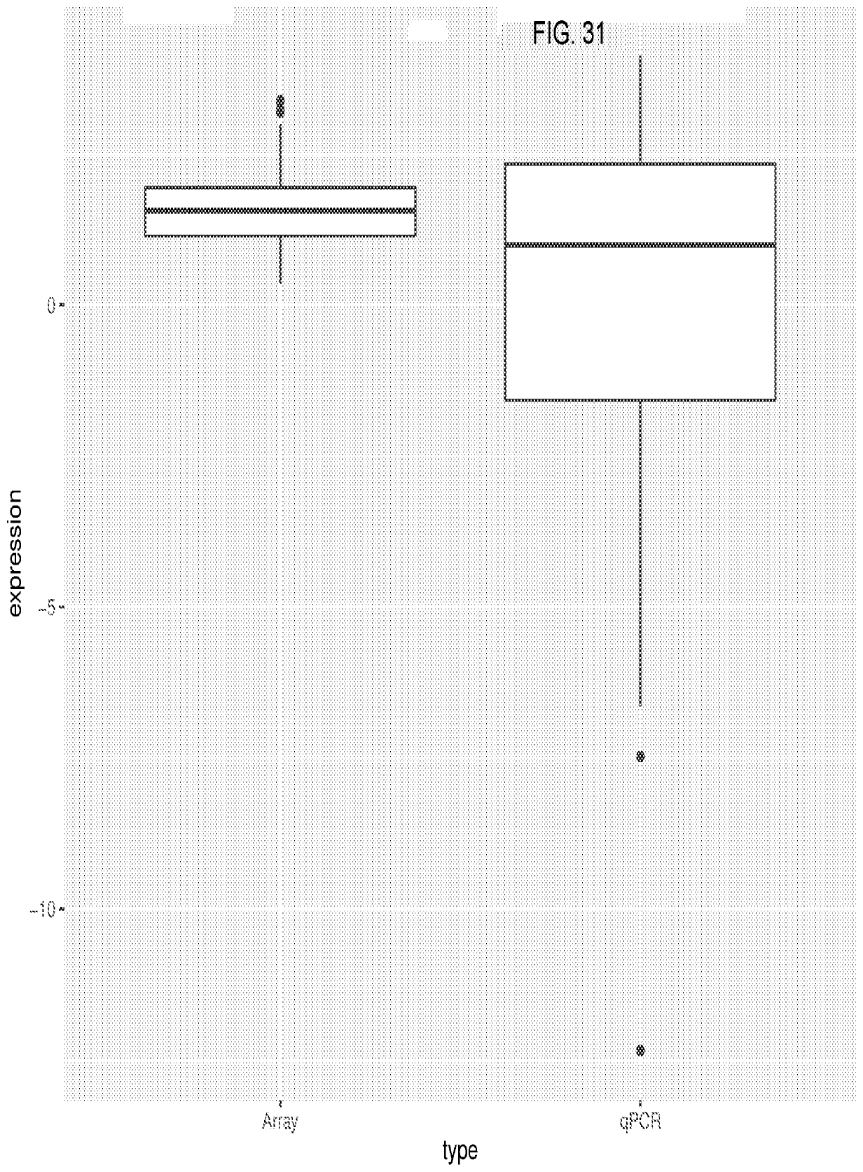

FIGS. 7-32.

The expression values of the each of 25 gene transcripts depicted in bar-whisker plot. The “expression” value (Y axis) represent the log (base 2) transformed expression values. X-axis or “Type” represents the assay platforms used to probe the samples, namely high throughput microarray (labeled as “array”) and qPCR.

FIG. 7 shows a bar-whisker plot related to ARG1A expression product.

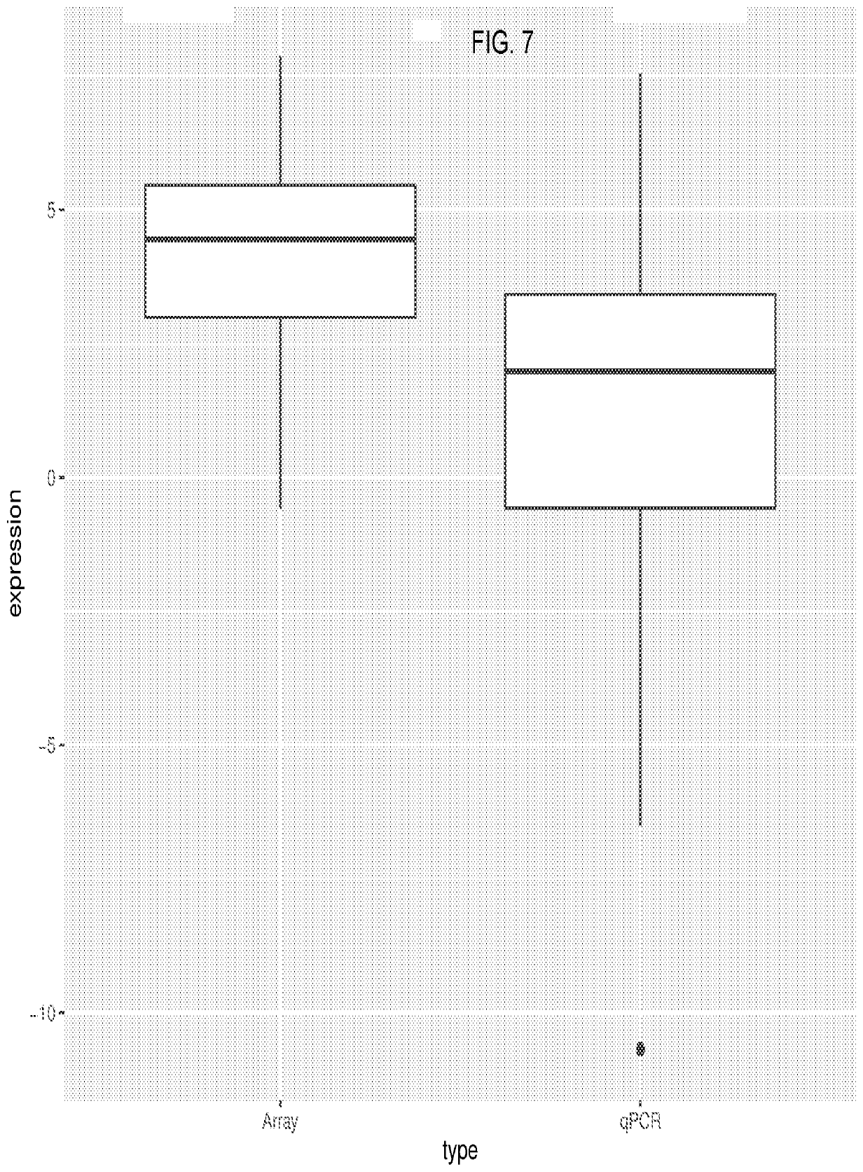

FIG. 8 shows a bar-whisker plot related to ARG1B expression product.

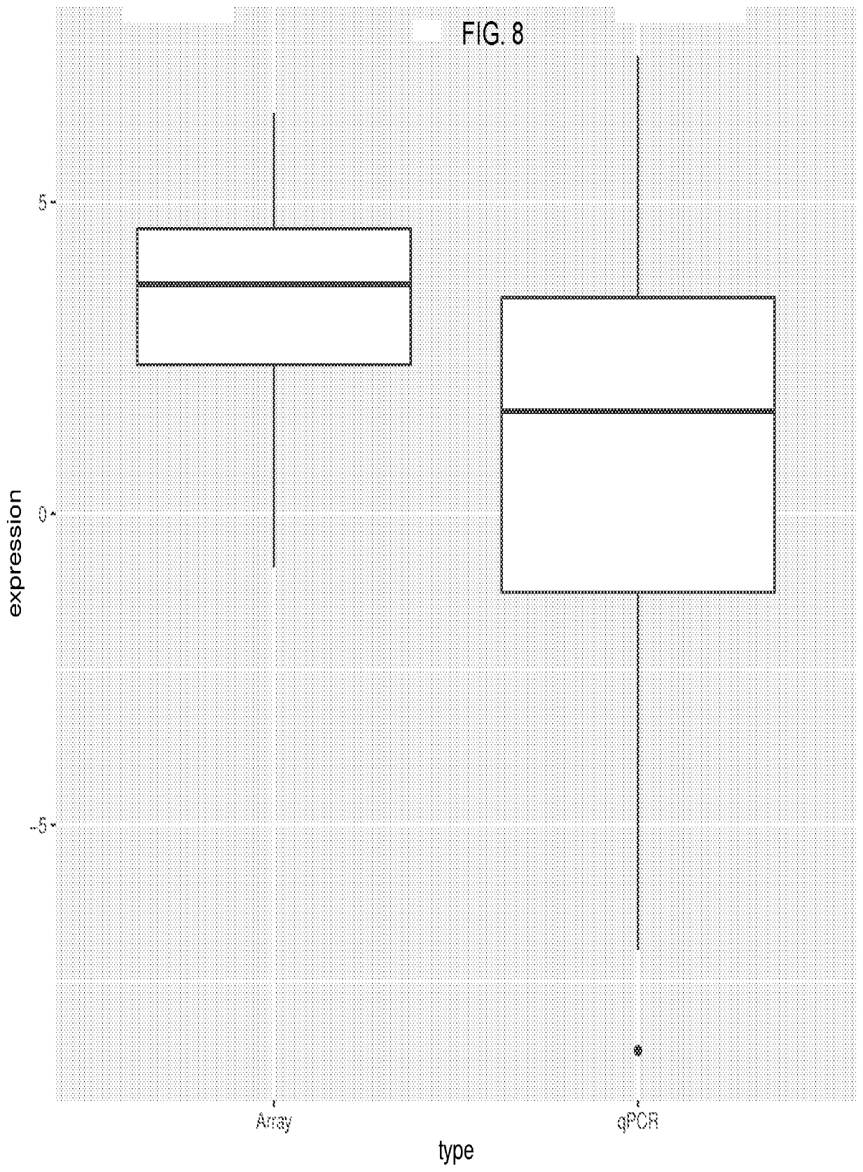

FIG. 9 shows a bar-whisker plot related to ATG2A expression product.

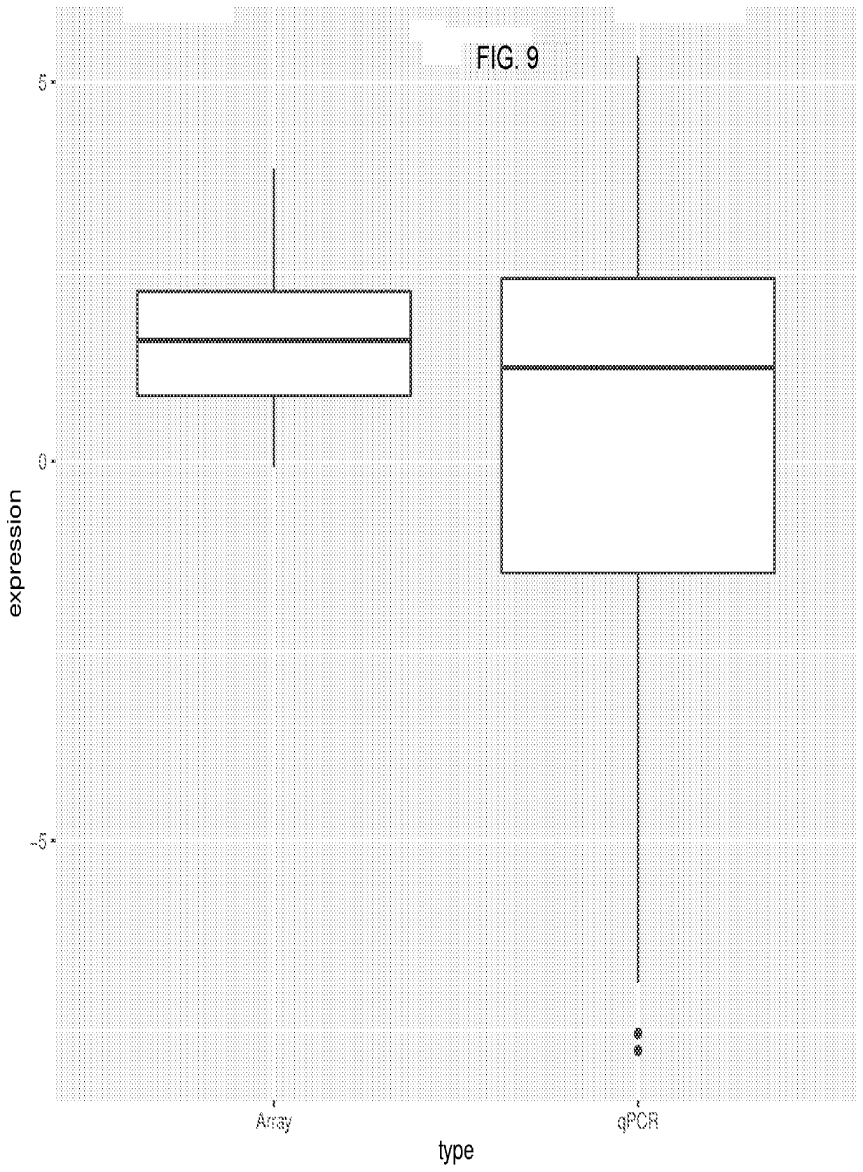

FIG. 10 shows a bar-whisker plot related to BCL2A1 expression product.

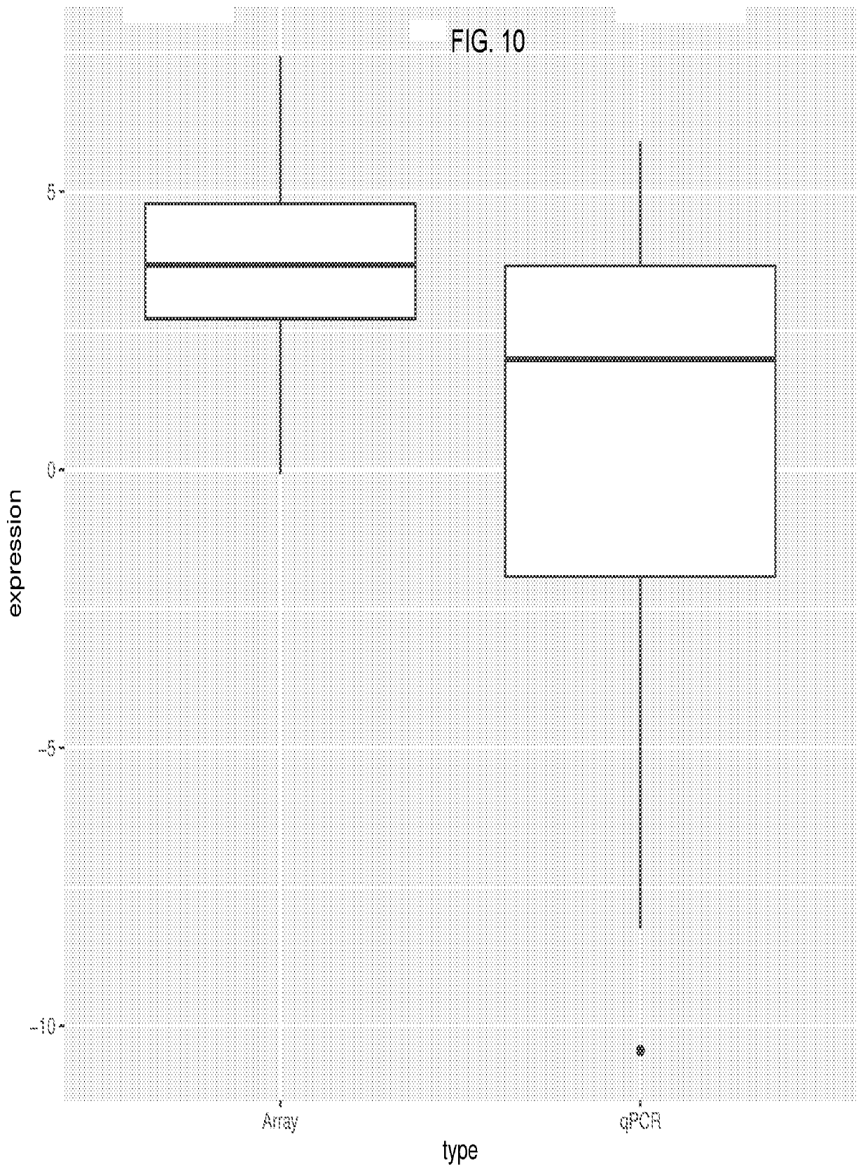

FIG. 11 shows a bar-whisker plot related to BMX expression product.

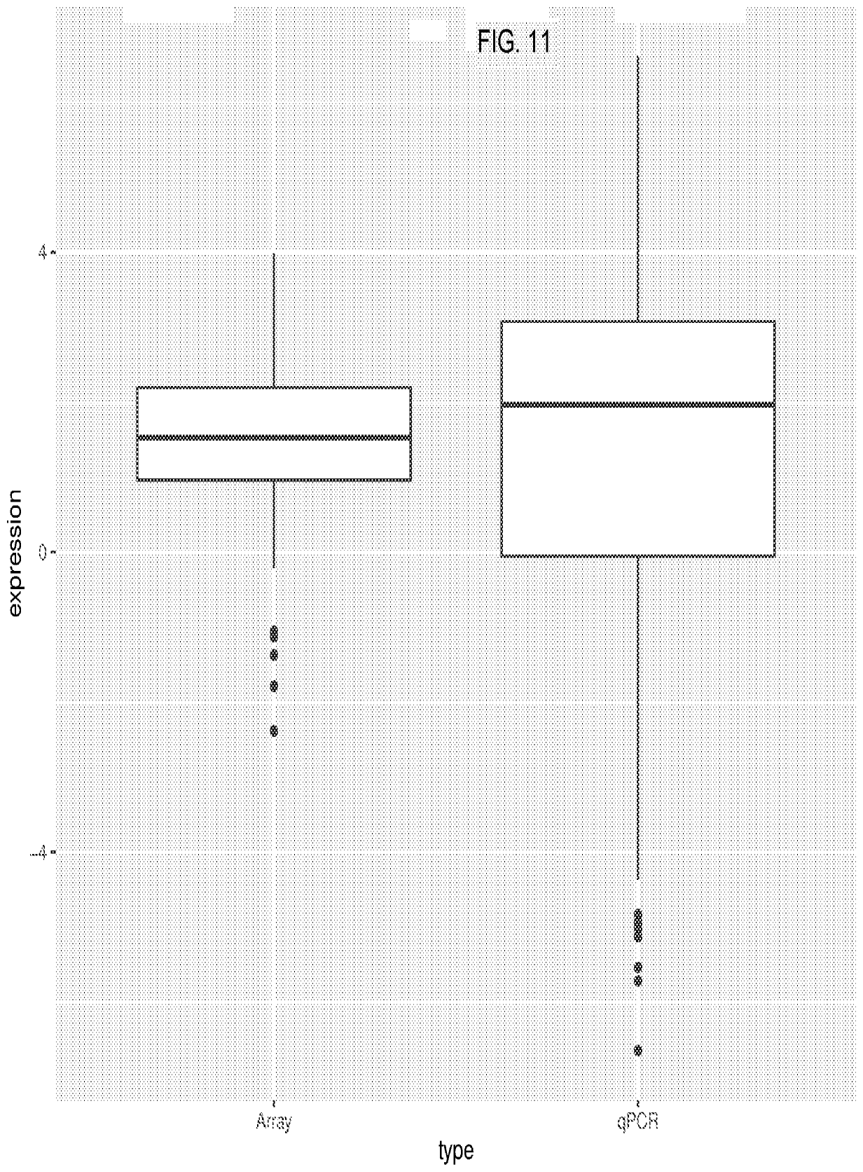

FIG. 12 shows a bar-whisker plot related to CD177 expression product.

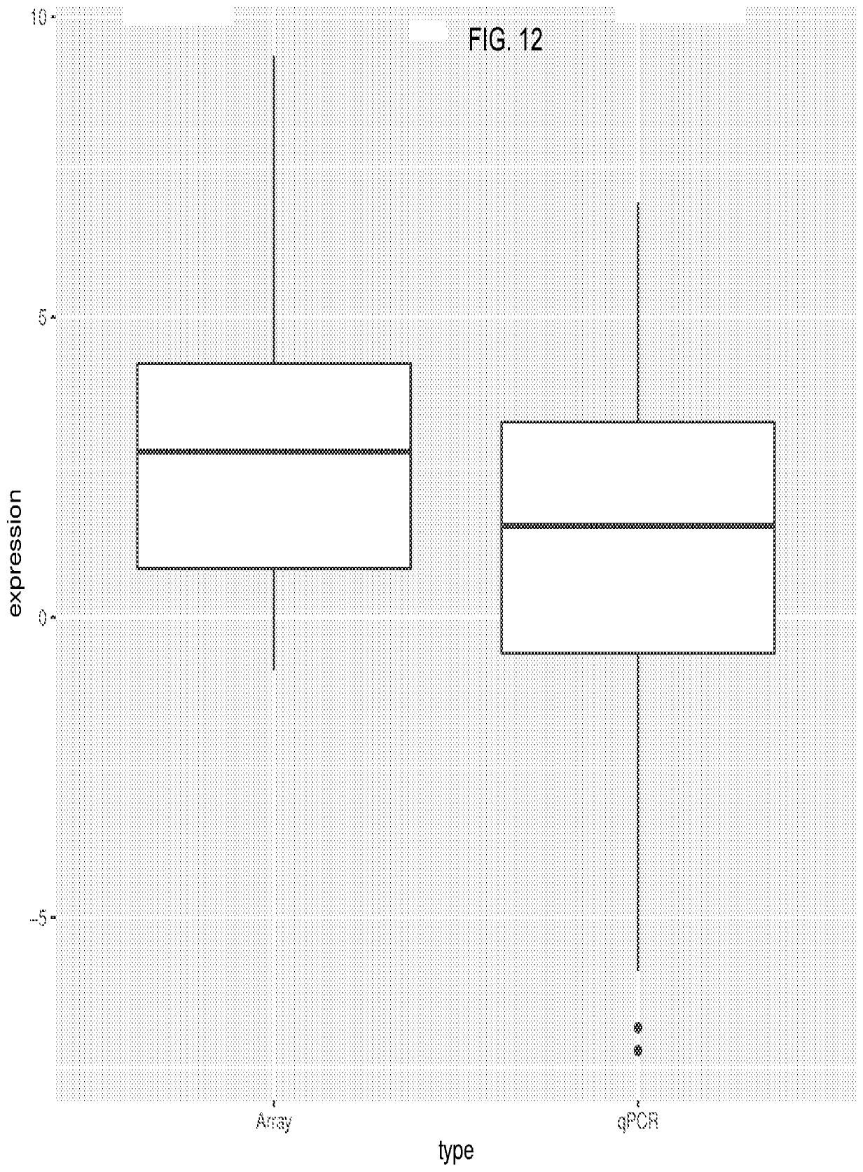

FIG. 13 shows a bar-whisker plot related to CEACAM4 expression product.

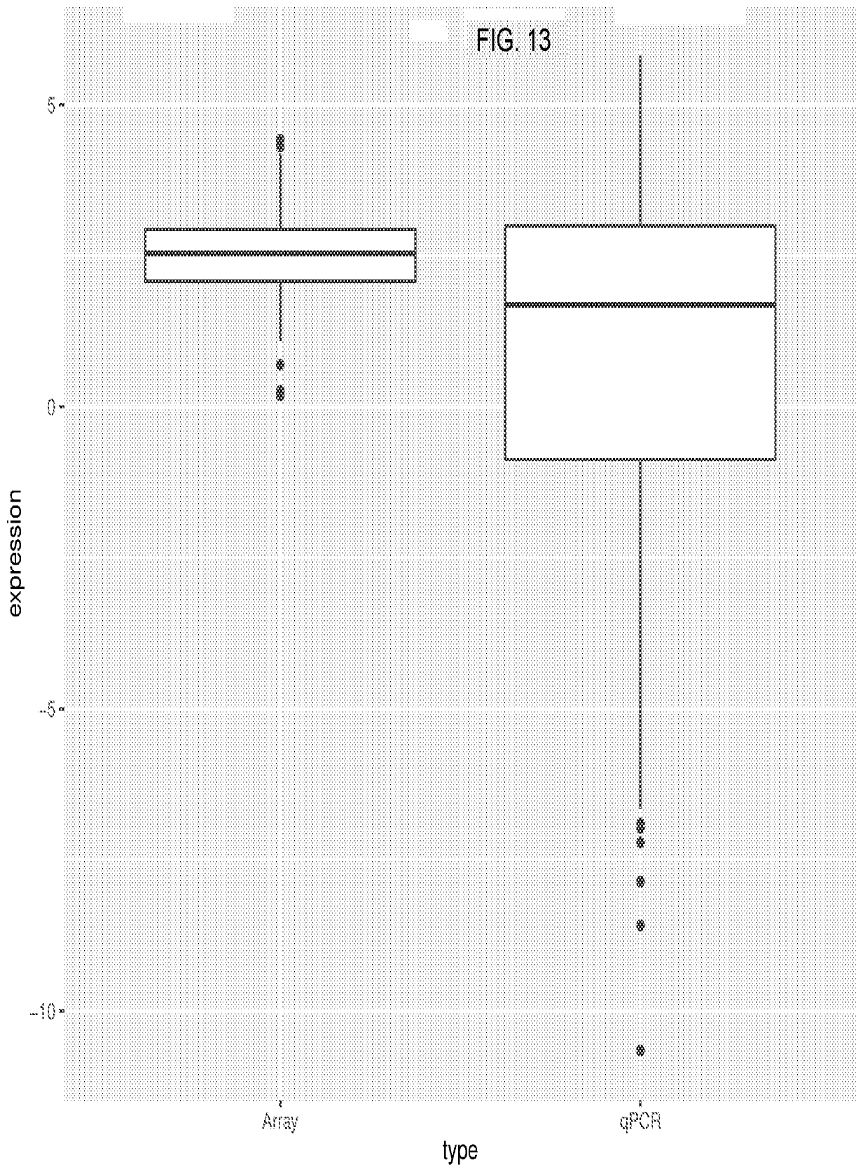

FIG. 14 shows a bar-whisker plot related to CLEC4D expression product.

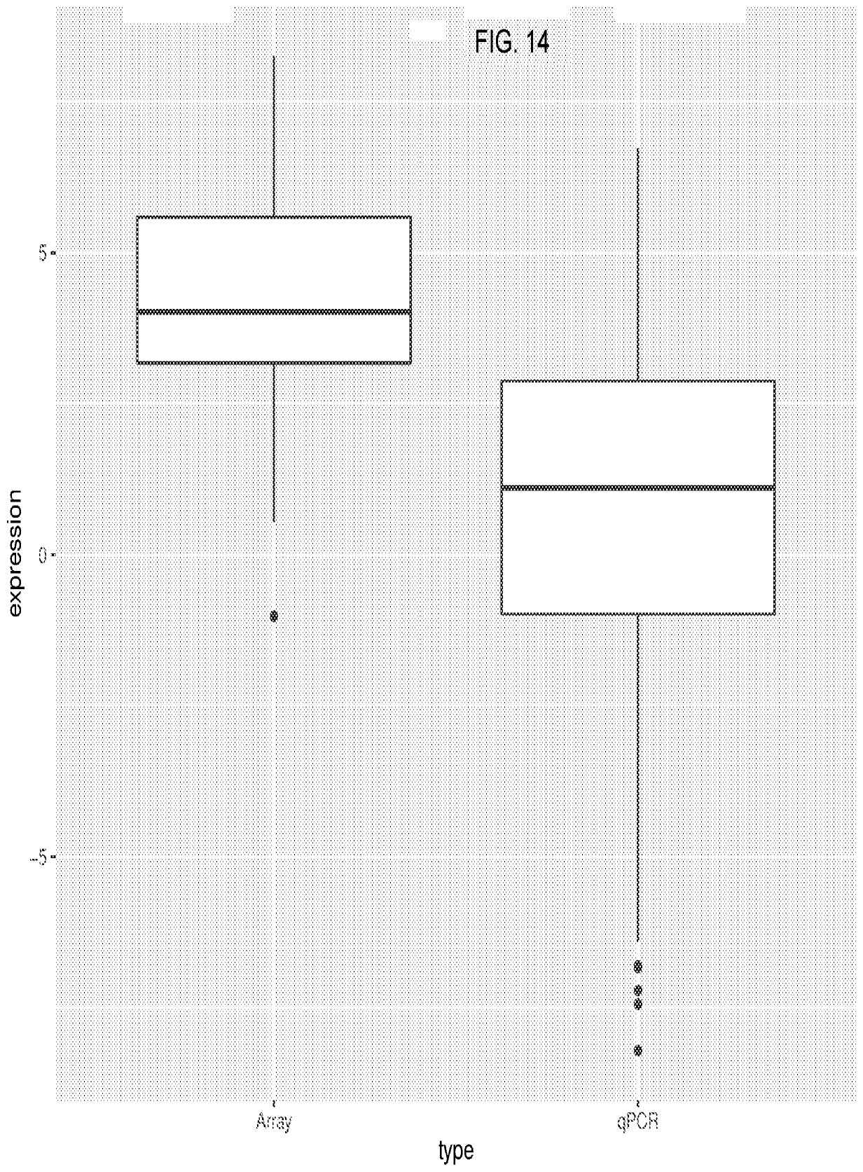

FIG. 15 shows a bar-whisker plot related to CLEC4D_A expression product.

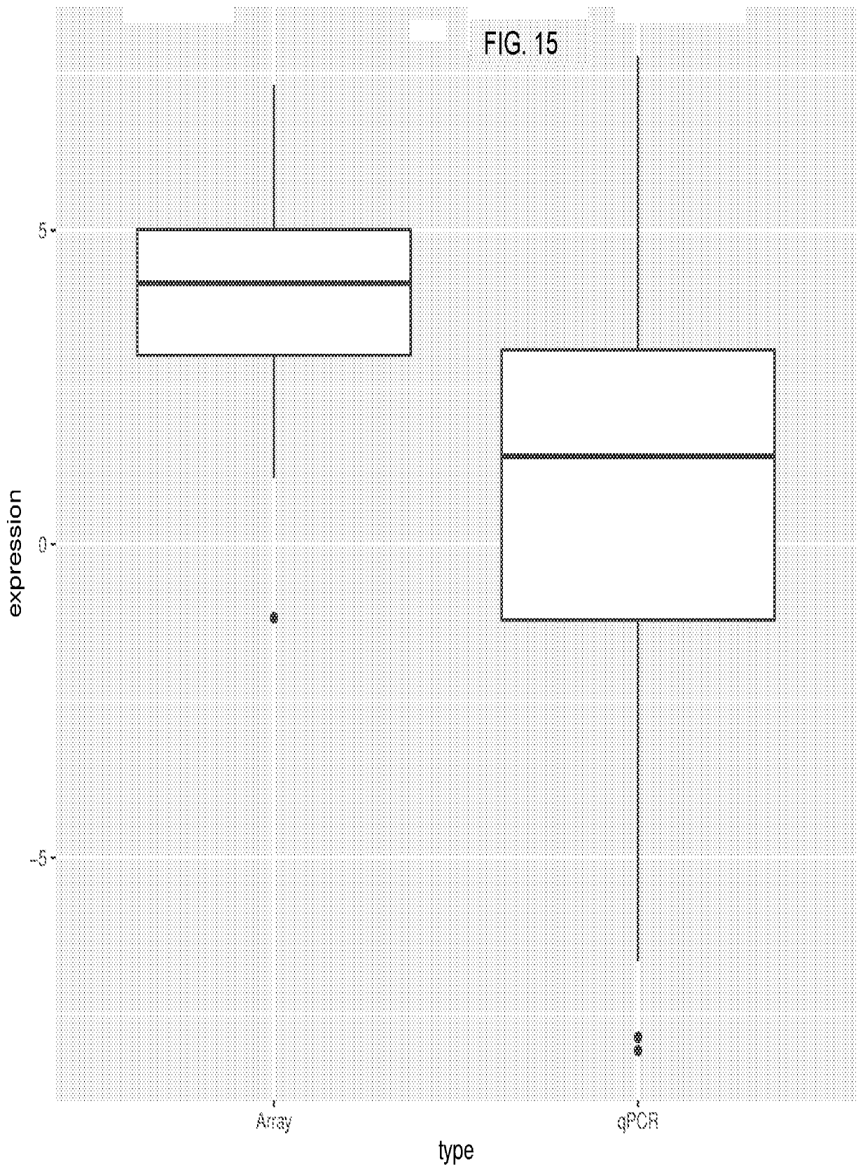

FIG. 16 shows a bar-whisker plot related to HP expression product.

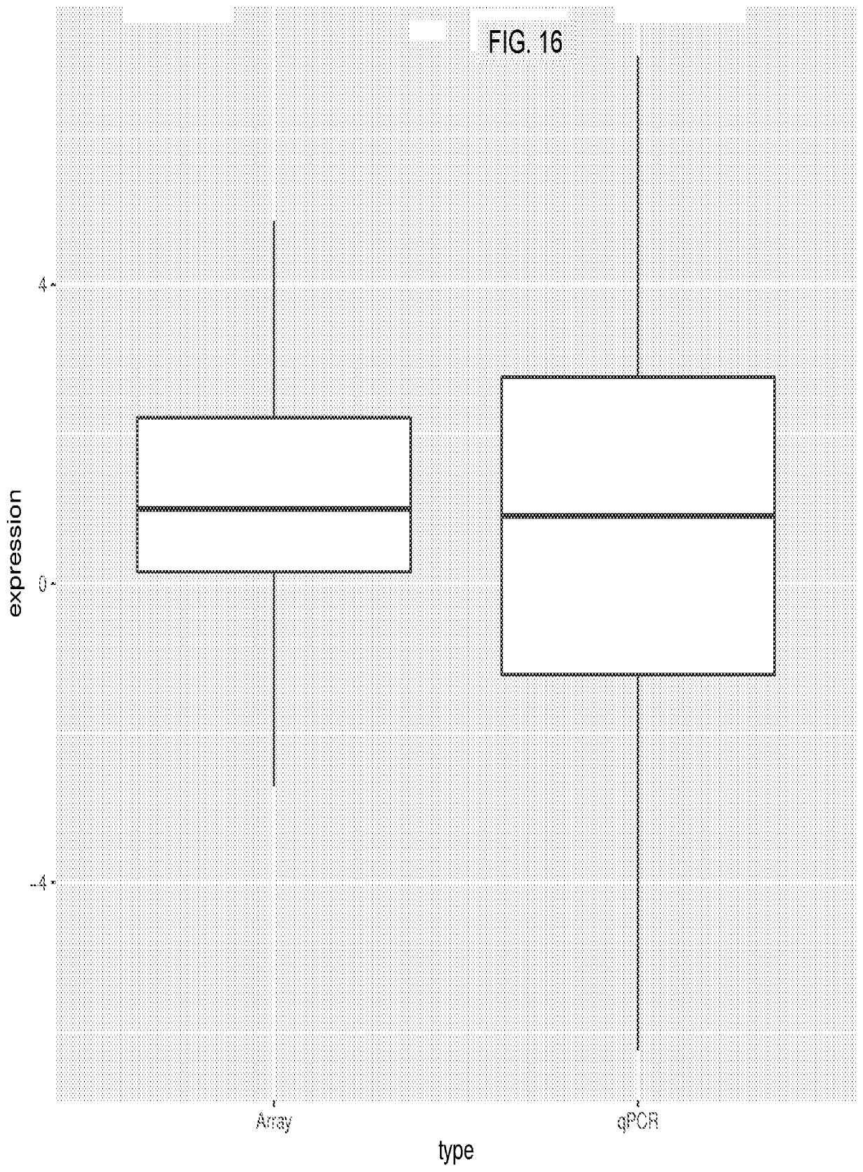

FIG. 17 shows a bar-whisker plot related to HPR expression product.

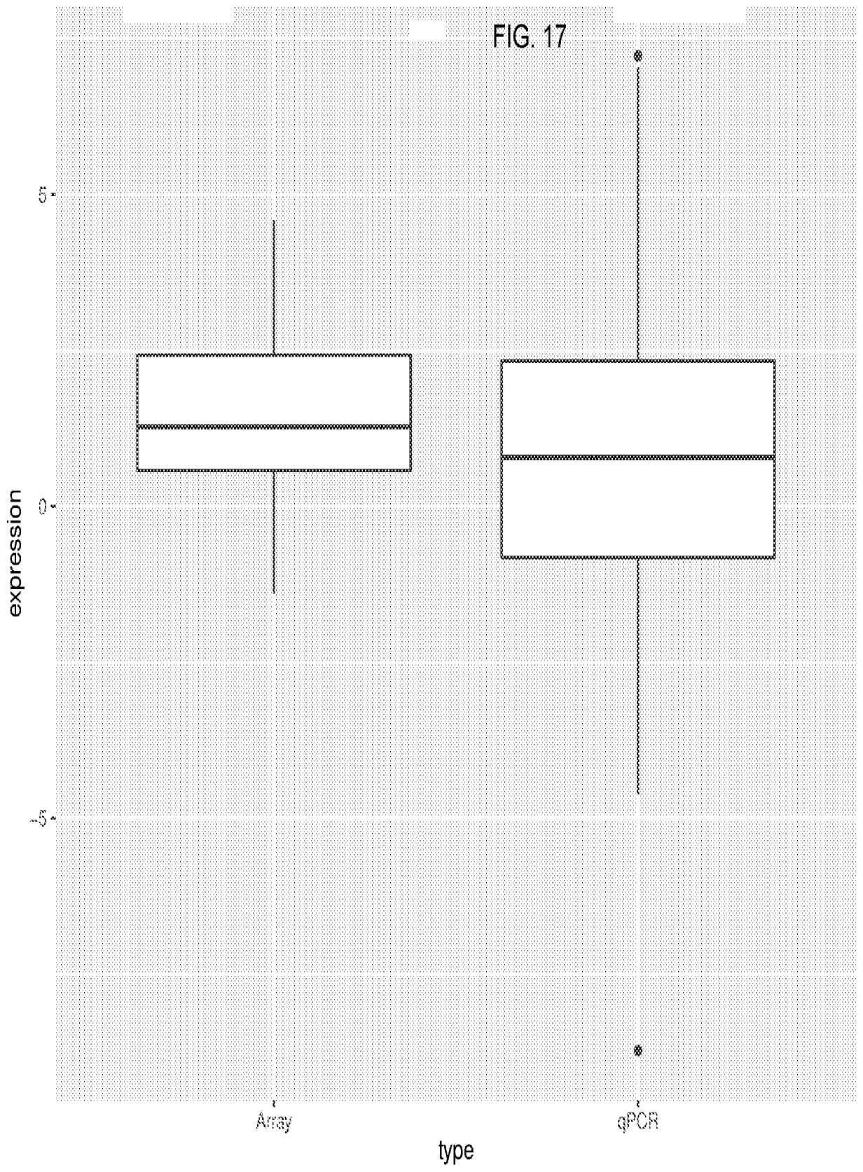

FIG. 18 shows a bar-whisker plot related to IL18R1 expression product.

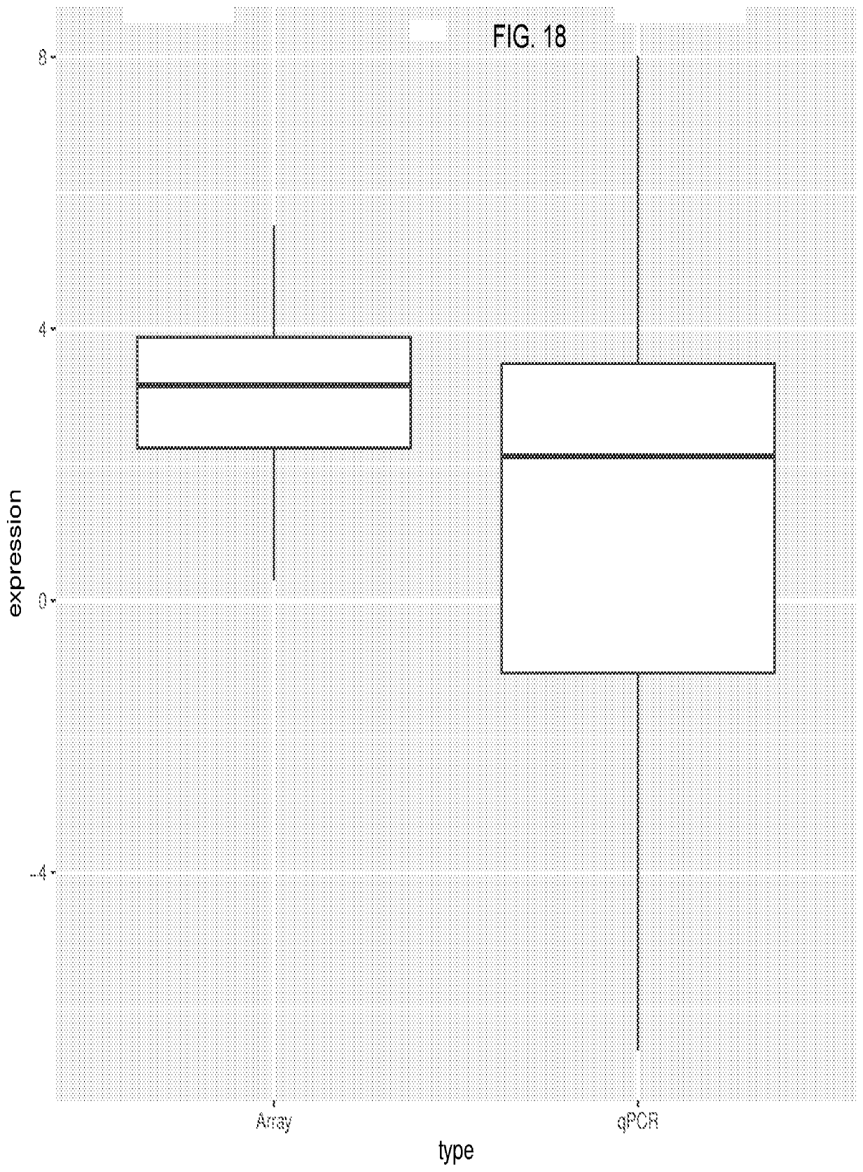

FIG. 19 shows a bar-whisker plot related to IL18RAP expression product.

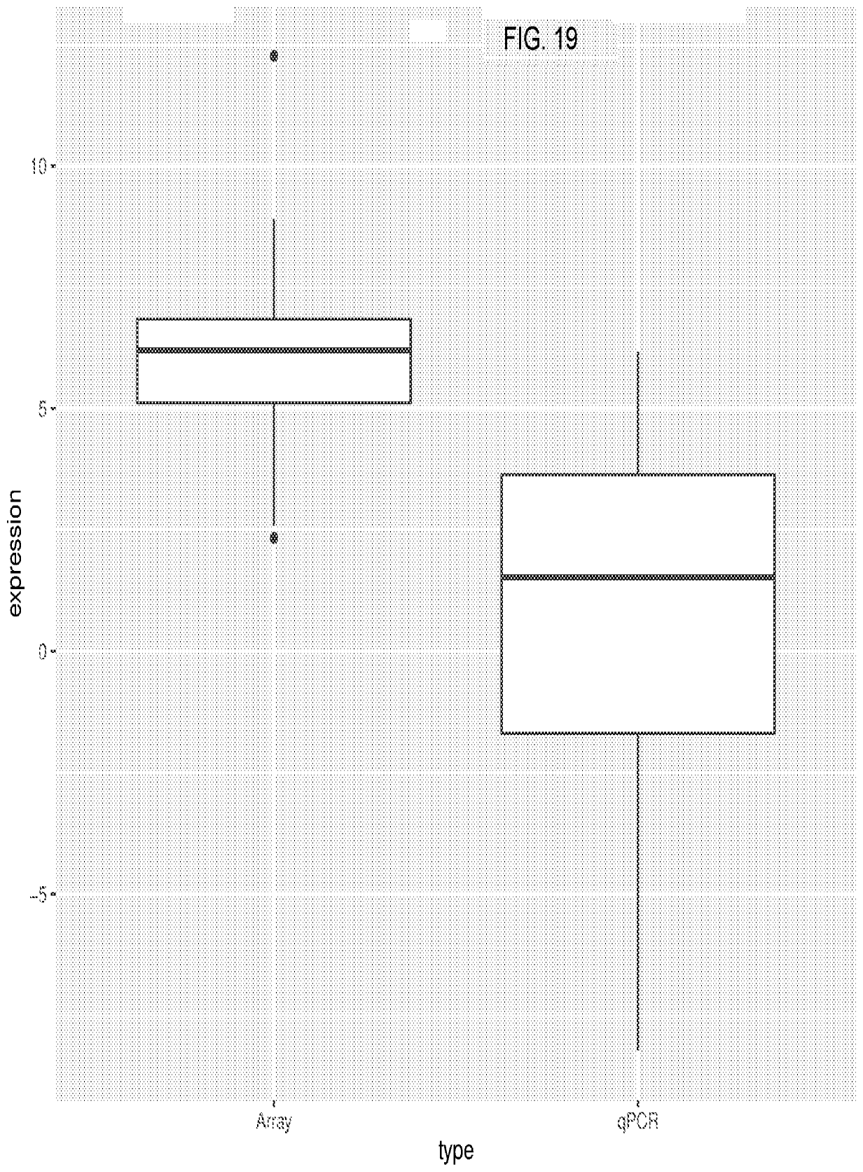

FIG. 20 shows a bar-whisker plot related to MMP8 expression product.

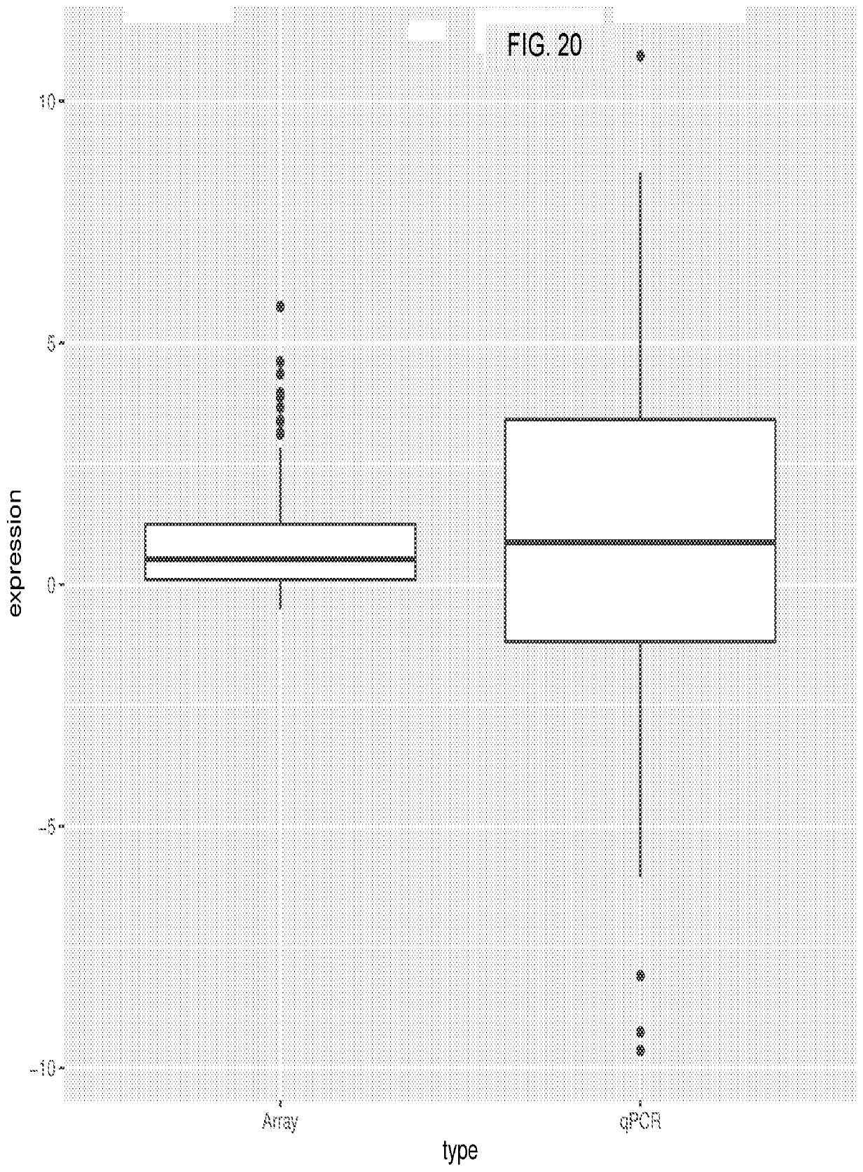

FIG. 21 shows a bar-whisker plot related to MS4A4A expression product.

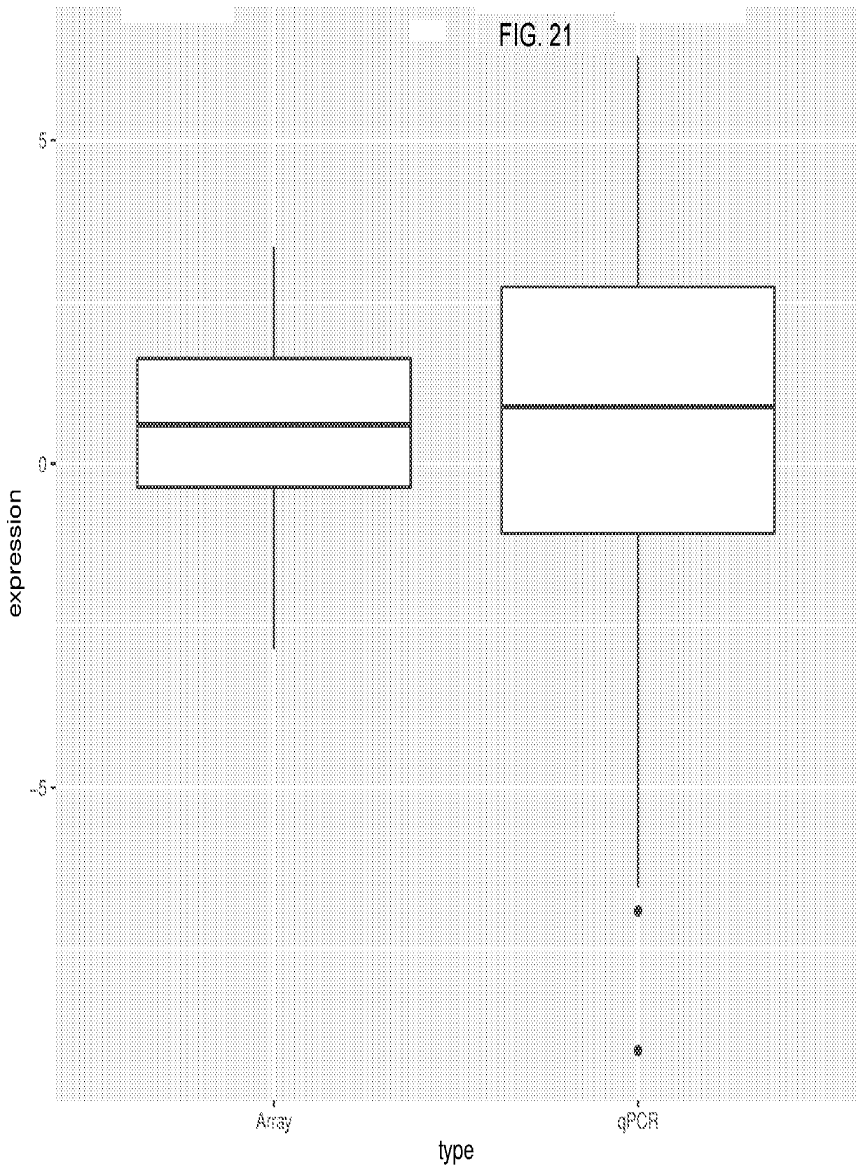

FIG. 22 shows a bar-whisker plot related to PADI4 expression product.

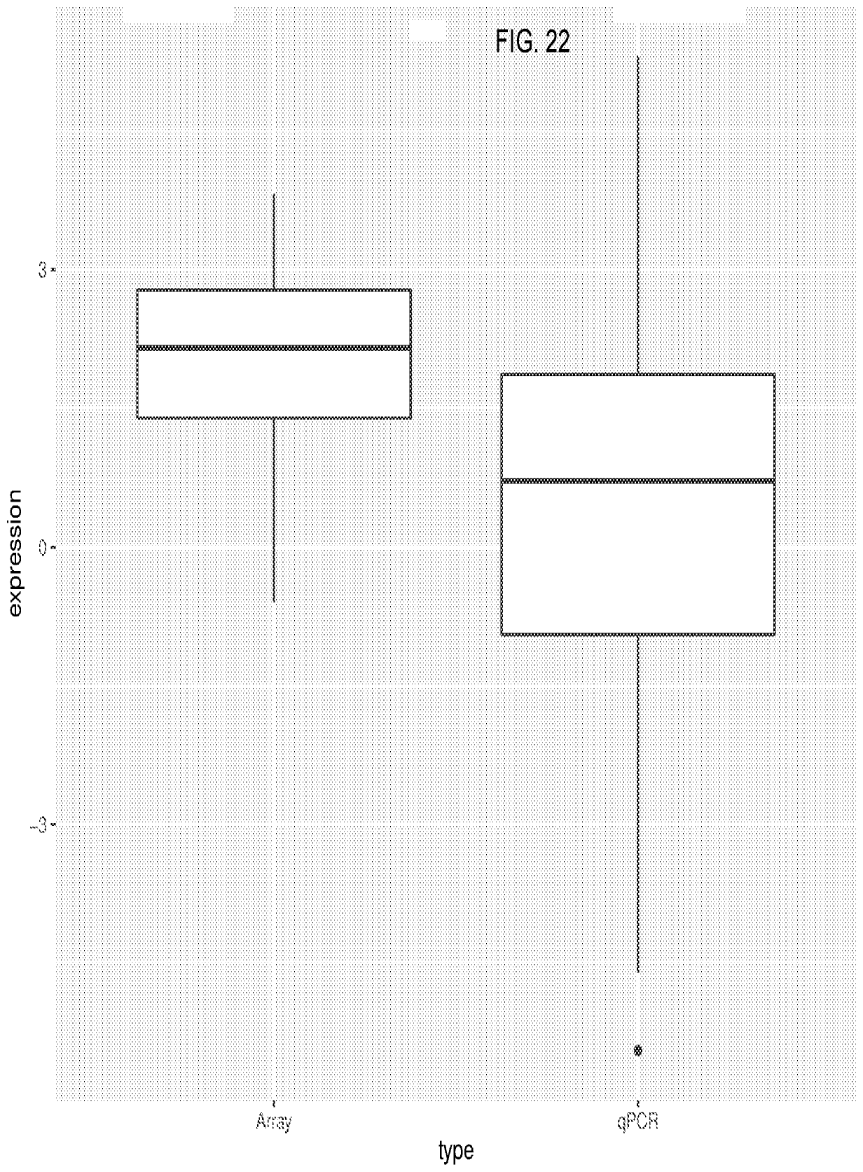

FIG. 23 shows a bar-whisker plot related to PFKFB2 expression product.

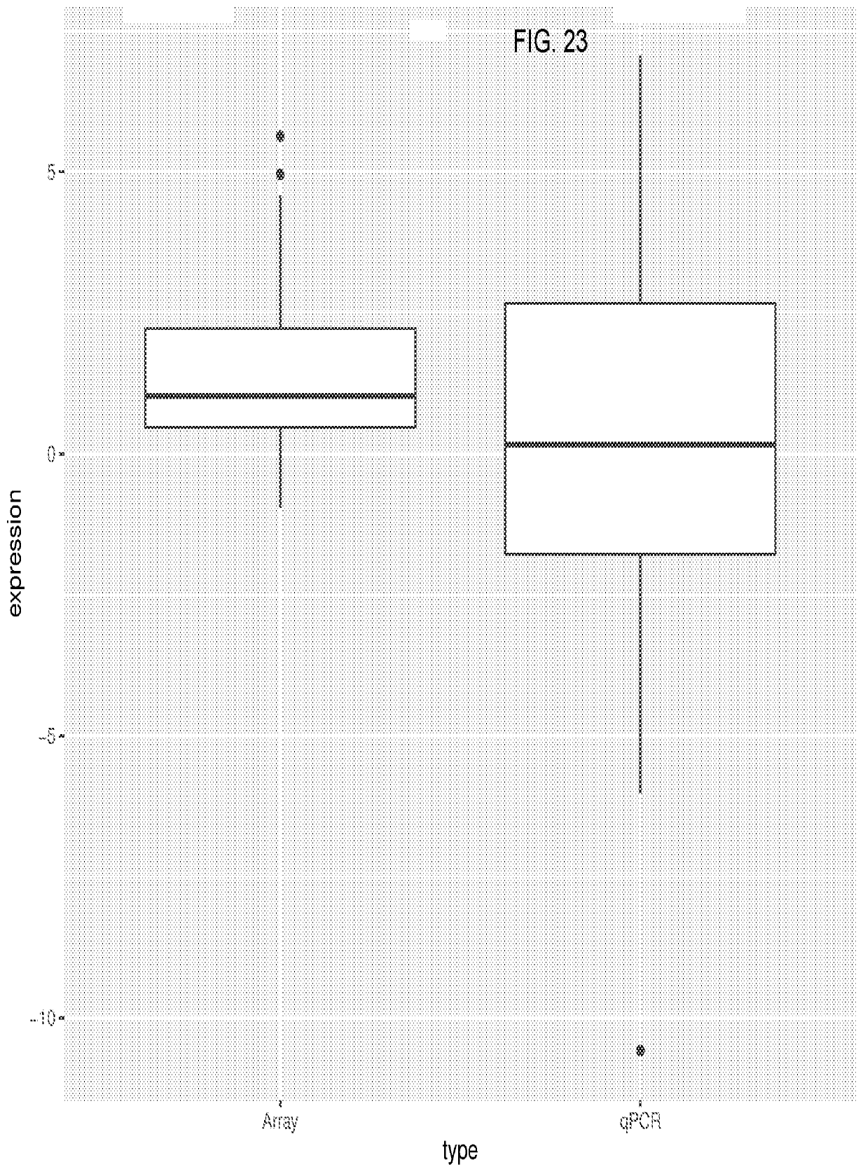

FIG. 24 shows a bar-whisker plot related to PLAC8_A expression product.

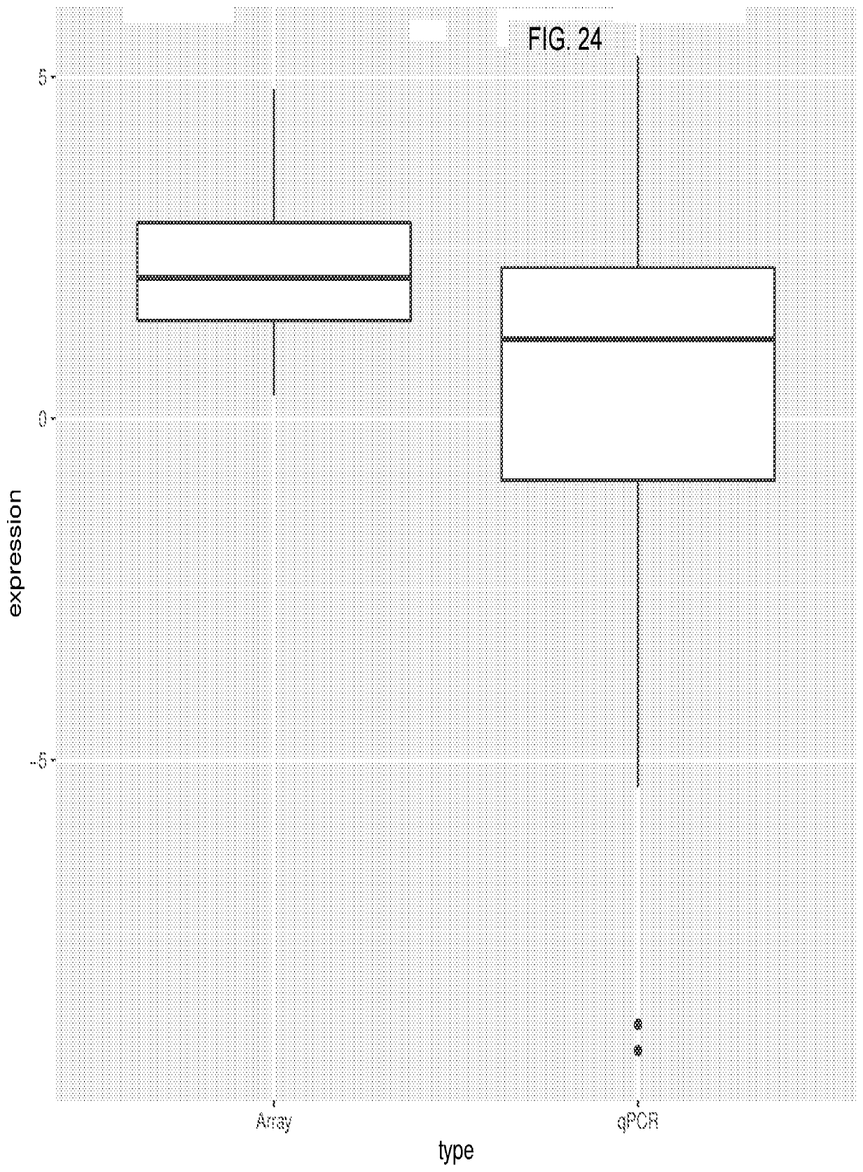

FIG. 25 shows a bar-whisker plot related to RNASE2 expression product.

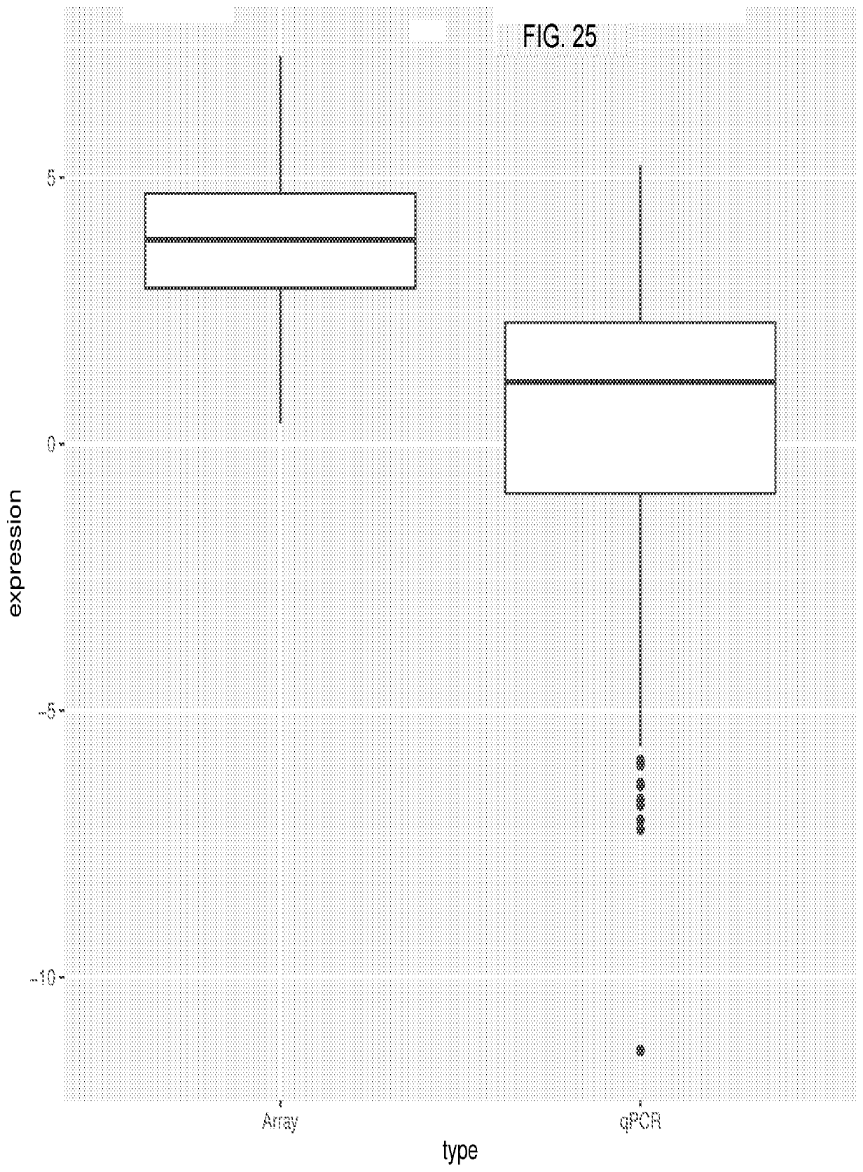

FIG. 26 shows a bar-whisker plot related to SIGLEC5 expression product.

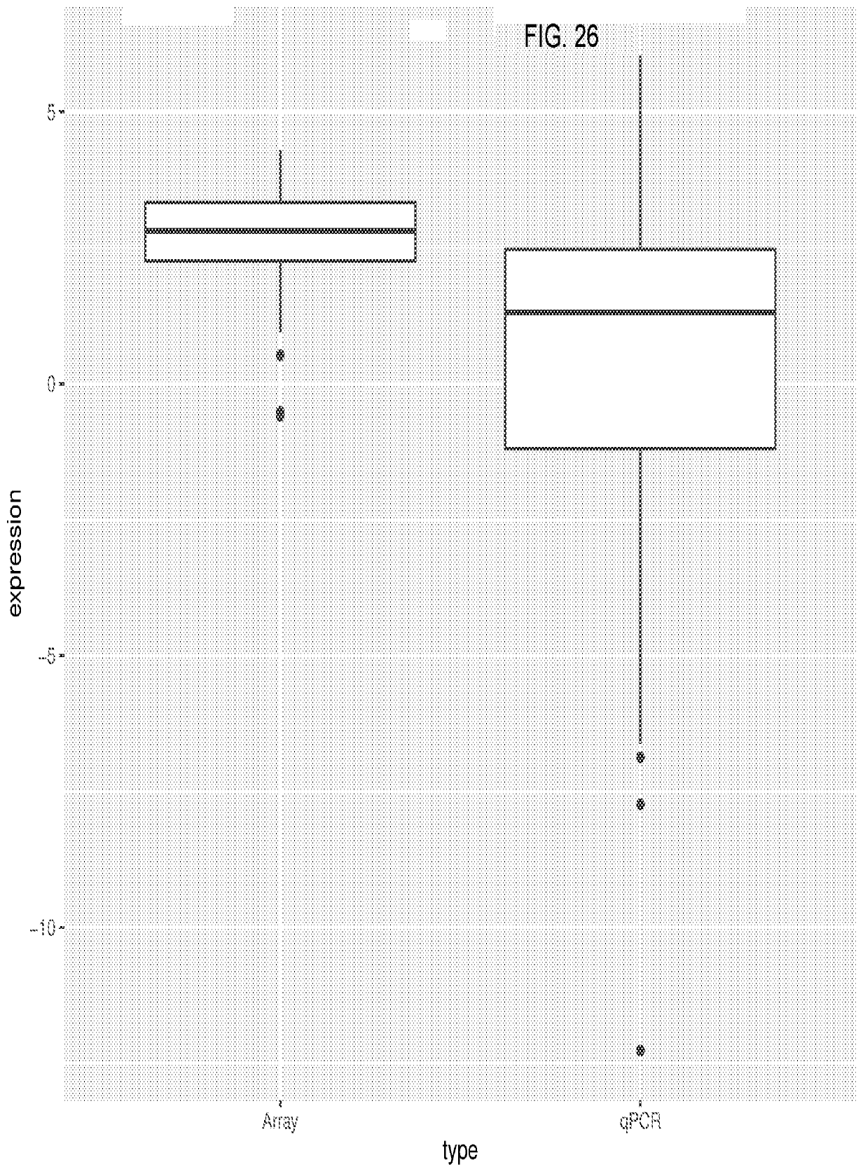

FIG. 27 shows a bar-whisker plot related to STOM expression product.

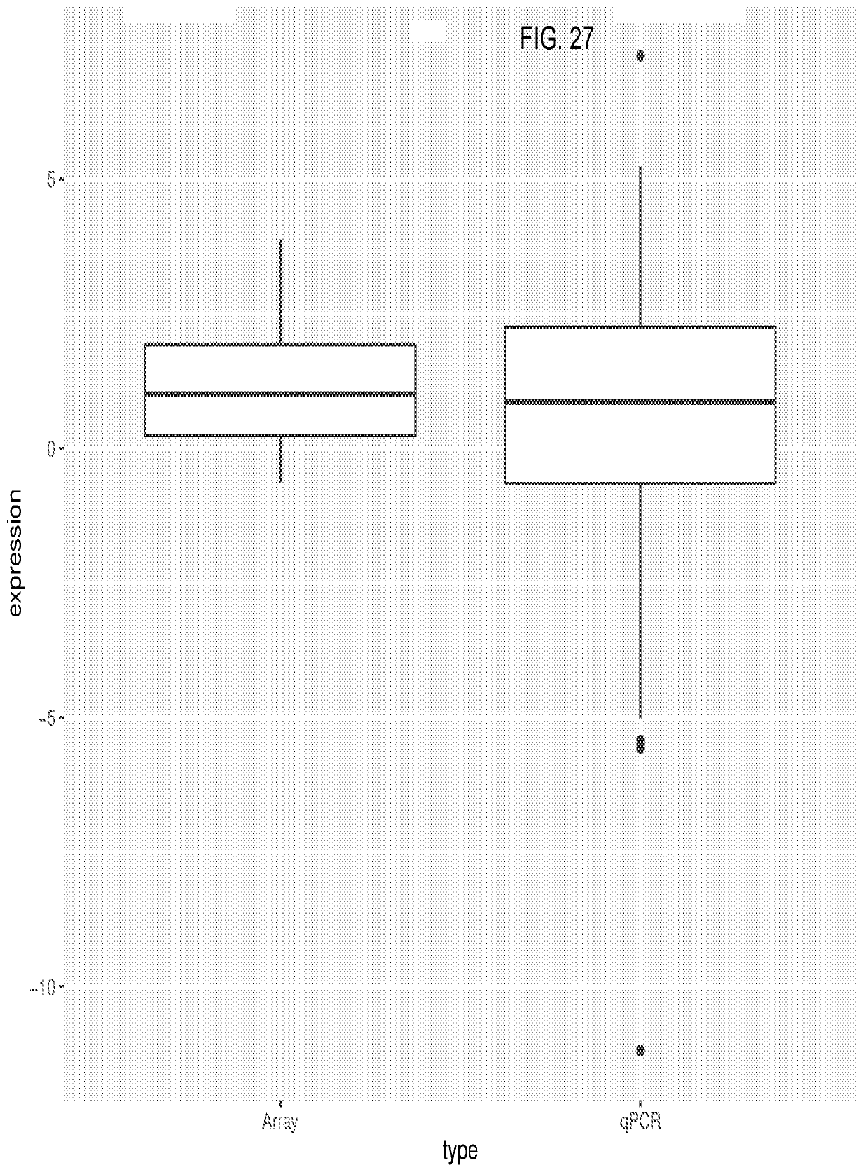

FIG. 28 shows a bar-whisker plot related to TDRD9 expression product.

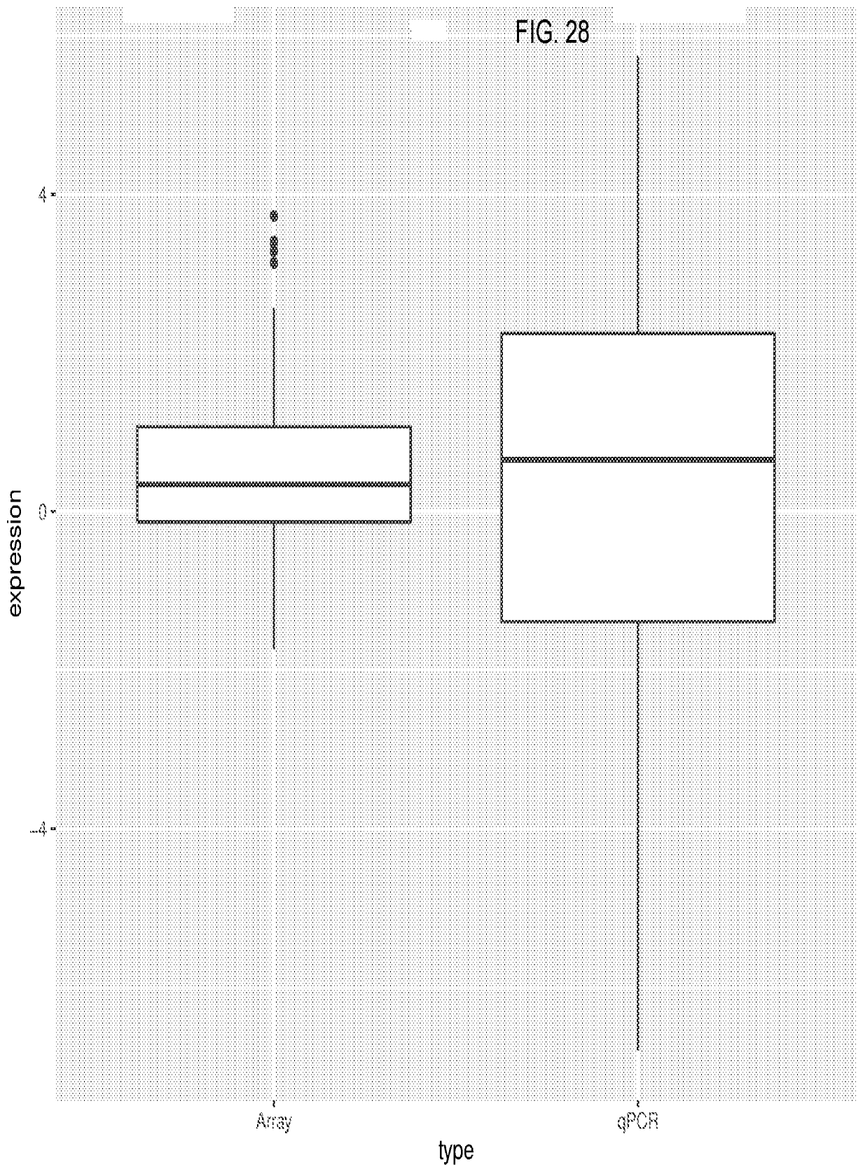

FIG. 29 shows a bar-whisker plot related to VINN1 expression product.

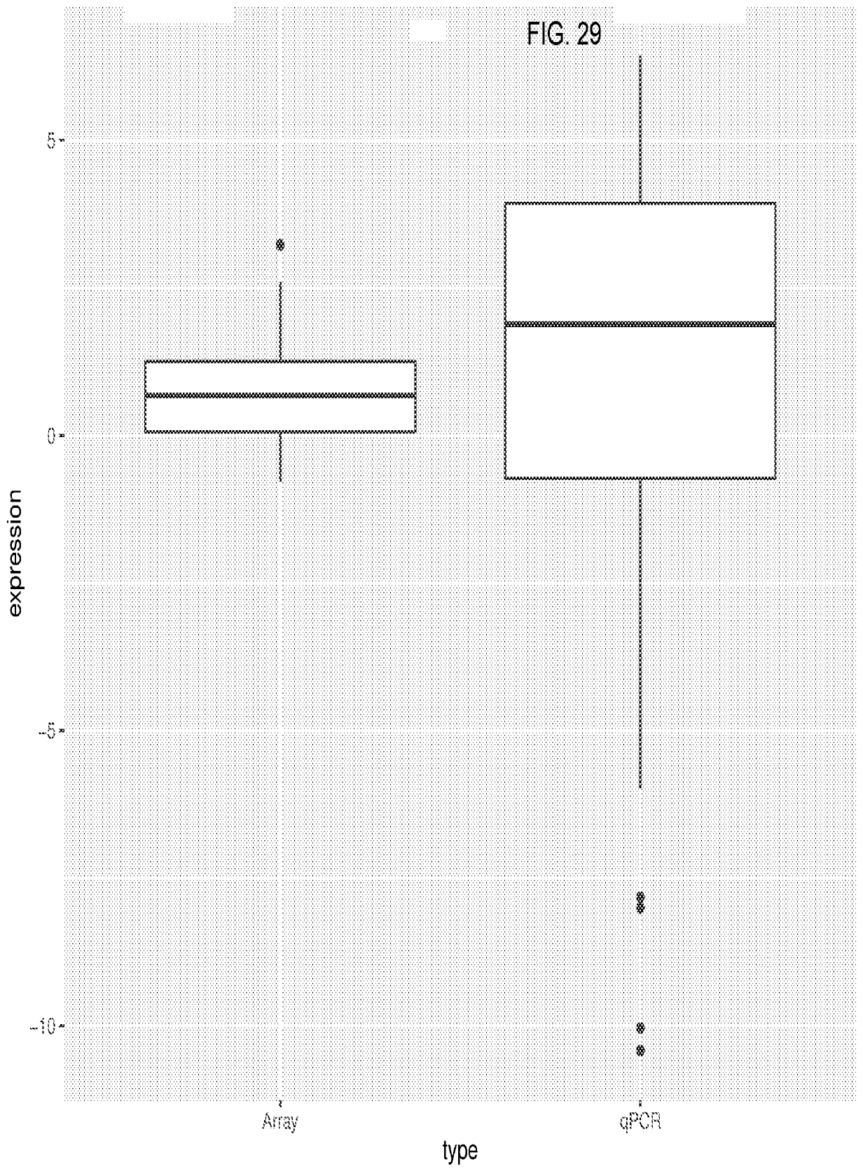

FIG. 30 shows a bar-whisker plot related to VINN1_2 expression product.

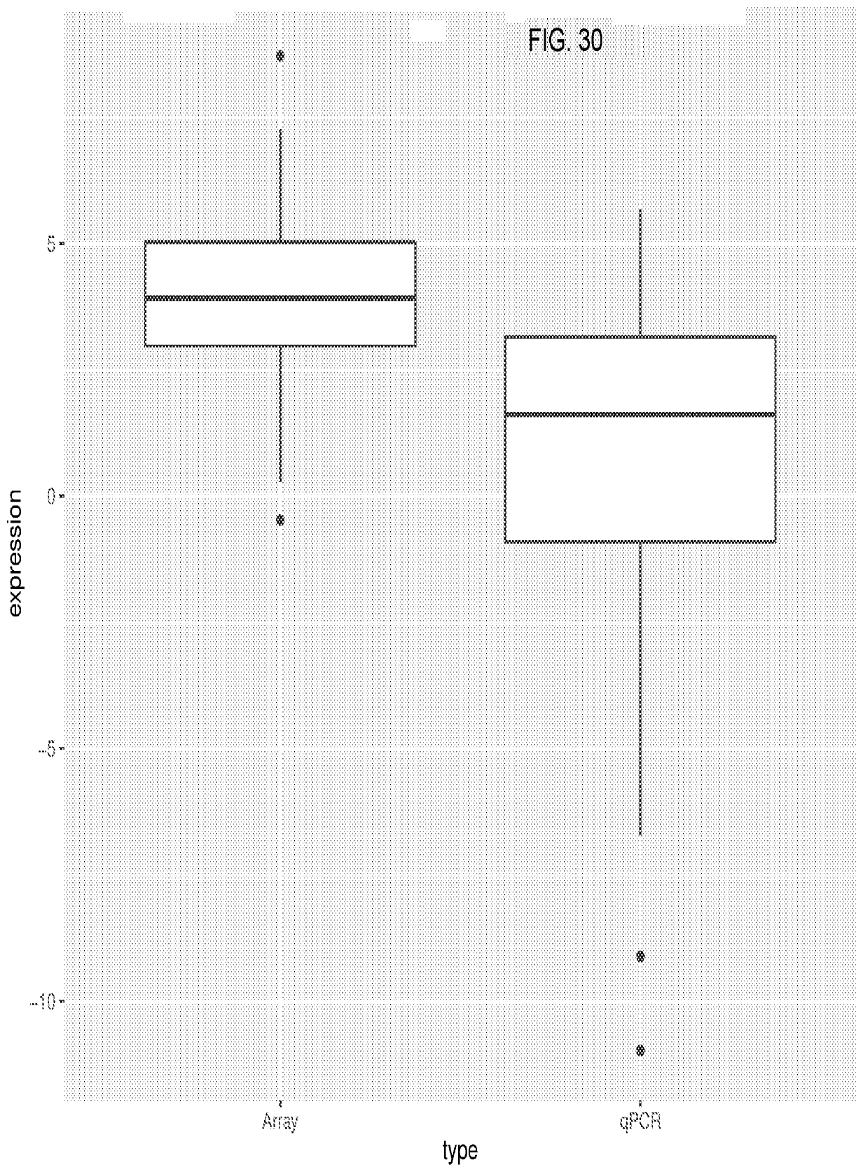

FIG. 31 shows a bar-whisker plot related to ZDHHC20 expression product.

### SEQUENCE LISTING

An XML file, named “15969-016PC0_ST26.xml”, 72 kb in size, and created on Aug. 30, 2022 is submitted with the application, and incorporated herein by reference.

### DETAILED DESCRIPTION

### Definitions

The term “amplifying” or “amplification” a nucleic acid sequence generally refers to the production of a plurality of nucleic acid copy molecules having that sequence from a target nucleic acid wherein primers hybridize to specific sites on the target nucleic acid molecules in order to provide an initiation site for extension by a polymerase, e.g., a DNA polymerase. Amplification can be carried out by any method generally known in the art, such as but not limited to: standard PCR, real-time PCR, long PCR, hot start PCR, qPCR, Reverse Transcription PCR and Isothermal Amplification.

As used herein, the term “AUC” refers to the Area Under the Curve, for example, of a ROC Curve. That value can assess the merit or performance of a test on a given sample population with a value of 1 representing a good test ranging down to 0.5 which means the test is providing a random response in classifying test subjects. Since the range of the AUC is only 0.5 to 1.0, a small change in AUC has greater significance than a similar change in a metric that ranges for 0 to 1 or 0 to 100%. When the % change in the AUC is given, it will be calculated based on the fact that the full range of the metric is 0.5 to 1.0. A variety of statistics packages can calculate AUC for a ROC curve, such as, JMP™ or Analyse-It™.

AUC can be used to compare the accuracy of the predictive model across the complete data range. Prediction models with greater AUC have, by definition, a greater capacity to classify unknowns correctly between the two groups of interest (disease and no disease).

As used herein, the term “biomarker” (or fragment thereof, or variant thereof) and their synonyms, which are used interchangeably, refer to molecules that can be evaluated in a sample and are associated with a physical condition. For example, markers include expressed genes or their products (e.g., proteins) or autoantibodies to those proteins that can be detected from human samples, such as blood, serum, solid tissue, and the like, that is associated with a physical or disease condition. Such biomarkers include, but are not limited to, biomolecules comprising nucleotides, amino acids, sugars, fatty acids, steroids, metabolites, polypeptides, proteins (such as, but not limited to, antigens and antibodies), carbohydrates, lipids, hormones, antibodies, regions of interest which serve as surrogates for biological molecules, combinations thereof (e.g., glycoproteins, ribonucleoproteins, lipoproteins) and any complexes involving any such biomolecules, such as, but not limited to, a complex formed between an antigen and an autoantibody that binds to an available epitope on said antigen. In a specific embodiment, the biomarker is an expression product of a gene.

The term “biomarker value” refers to a value measured or derived for at least one corresponding biomarker of the biological subject and which is typically at least partially indicative of a concentration of the biomarker in a sample taken from the subject. Thus, the biomarker values could be measured biomarker values, which are values of biomarkers measured for the subject, or alternatively could be derived biomarker values, which are values that have been derived from one or more measured biomarker values, for example by applying a function to the one or more measured biomarker values.

Biomarker values can be of any appropriate form depending on the manner in which the values are determined. For example, the biomarker values could be determined using high-throughput technologies such as mass spectrometry, sequencing platforms, array and hybridization platforms, immunoassays, flow cytometry, or any combination of such technologies and in one preferred example, the biomarker values relate to a level of activity or abundance of an expression product or other measurable molecule, quantified using a technique such as PCR, sequencing or the like. In this case, the biomarker values can be in the form of amplification amounts, or cycle times, which are a logarithmic representation of the concentration of the biomarker within a sample, as will be appreciated by persons skilled in the art and as will be described in more detail below.

As used herein, the term “detecting” refers to observing a signal from a label moiety to indicate the presence of a biomarker in the sample. Any method known in the art for detecting a particular detectable moiety can be used for detection. Exemplary detection methods include, but are not limited to, spectroscopic, photochemical, biochemical, immunochemical, electrical, optical or chemical methods.

The term “effective amount” refers to an amount of a therapeutic agent that is sufficient to exert a physiological effect in the subject.

The term “expression product” refers to a polynucleotide expression product (e.g. transcript) or a polypeptide expression product (e.g. protein).

The term “labeling probe” generally, according to various embodiments, refers to a molecule used in an amplification reaction, typically for quantitative or qPCR analysis, as well as end-point analysis. Such labeling probes may be used to monitor the amplification of the target polynucleotide. In some embodiments, oligonucleotide labeling probes present in an amplification reaction are suitable for monitoring the amount of amplicon(s) produced as a function of time. Such oligonucleotide labeling probes include, but are not limited to, 5′-exonuclease assay TaqMan® labeling probes described herein (see also U.S. Pat. No. 5,538,848), various stem-loop molecular beacons (see e.g., U.S. Pat. Nos. 6,103,476 and 5,925,517 and Tyagi and Kramer, 1996, Nature Biotechnology 14:303-308), stemless or linear beacons (see, e.g., WO 99/21881), PNA Molecular Beacons™ (see, e.g., U.S. Pat. Nos. 6,355,421 and 6,593,091), linear PNA beacons (see, e.g., Kubista et al., 2001, SPIE 4264:53-58), non-FRET labeling probes (see, e.g., U.S. Pat. No. 6,150,097), Sunrise®/Amplifluor® labeling probes (U.S. Pat. No. 6,548,250), stem-loop and duplex Scorpion™ labeling probes (Solinas et al., 2001, Nucleic Acids Research 29: E96 and U.S. Pat. No. 6,589,743), bulge loop labeling probes (U.S. Pat. No. 6,590,091), pseudo knot labeling probes (U.S. Pat. No. 6,589,250), cyclicons (U.S. Pat. No. 6,383,752), MGB Eclipse™ probe (Epoch Biosciences), hairpin labeling probes (U.S. Pat. No. 6,596,490), peptide nucleic acid (PNA) light-up labeling probes, self-assembled nanoparticle labeling probes, and ferrocene-modified labeling probes described, for example, in U.S. Pat. No. 6,485,901; Mhlanga et al., 2001, Methods 25:463-471; Whitcombe et al., 1999, Nature Biotechnology. 17:804-807; Isacsson et al., 2000, Molecular Cell Labeling probes. 14:321-328; Svanvik et al., 2000, Anal Biochem. 281:26-35; Wolffs et al., 2001, Biotechniques 766:769-771; Tsourkas et al., 2002, Nucleic Acids Research. 30:4208-4215; Riccelli et al., 2002, Nucleic Acids Research 30:4088-4093; Zhang et al., 2002 Shanghai. 34:329-332; Maxwell et al., 2002, J. Am. Chem. Soc. 124:9606-9612; Broude et al., 2002, Trends Biotechnol. 20:249-56; Huang et al., 2002, Chem Res. Toxicol. 15:118-126; and Yu et al., 2001, J. Am. Chem. Soc 14:11155-11161. Labeling probes can also comprise black hole quenchers (Biosearch), Iowa Black (IDT), QSY quencher (Molecular Labeling probes), and Dabsyl and Dabcel sulfonate/carboxylate Quenchers (Epoch). Labeling probes can also comprise two labeling probes, wherein for example a fluorophore is on one probe, and a quencher on the other, wherein hybridization of the two labeling probes together on a target quenches the signal, or wherein hybridization on target alters the signal signature via a change in fluorescence. Labeling probes can also comprise sulfonate derivatives of fluorescenin dyes with a sulfonic acid group instead of the carboxylate group, phosphoramidite forms of fluorescein, phosphoramidite forms of CY 5 (available for example from Amersham).

As used herein “machine learning” refers to algorithms that give a computer the ability to learn without being explicitly programmed including algorithms that learn from and make predictions about data. Machine learning algorithms include, but are not limited to, decision tree learning, artificial neural networks (ANN) (also referred to herein as a “neural net”), deep learning neural network, support vector machines, rule base machine learning, random forest, logistic regression, pattern recognition algorithms, etc. For the purposes of clarity, algorithms such as linear regression or logistic regression can be used as part of a machine learning process. However, it is understood that using linear regression or another algorithm as part of a machine learning process is distinct from performing a statistical analysis such as regression with a spreadsheet program such as Excel. The machine learning process has the ability to continually learn and adjust the classifier model as new data becomes available and does not rely on explicit or rules-based programming. Statistical modeling relies on finding relationships between variables (e.g., mathematical equations) to predict an outcome. FIG. 6 provides examples of a machine learning algorithm, that involve K-fold cross validation and/or Random Single Bin Multiple Repeats (RSBMR) statistical processes.

The term “sample” as used herein includes any biological specimen obtained from a patient. Samples include, without limitation, whole blood, plasma, serum, red blood cells, white blood cells (e.g., peripheral blood mononuclear cells), cord blood, ductal lavage fluid, nipple aspirate, lymph, bone marrow aspirate, saliva, urine, stool (i.e., feces), sputum, bronchial lavage fluid, tears, fine needle aspirate, any other bodily fluid, a tissue such as a biopsy of a tumor (e.g., needle biopsy) or a lymph node, and cellular extracts thereof. In some embodiments, the sample is whole blood or a fractional component thereof such as plasma, serum, or a cell pellet.

As used herein, the term “sepsis” refers to organ dysfunction caused by a dysregulated host response to an infection’, e.g., bacterial infection.

As used herein, the term “subject” or “patient” are used interchangeably herein to refer to a human or non-human mammal or animal. Non-human mammals include livestock animals, companion animals, laboratory animals, and non-human primates. Non-human subjects also specifically include, without limitation, chickens, horses, cows, pigs, goats, dogs, cats, guinea pigs, hamsters, mink, and rabbits. In some embodiments, a subject is a human burn patient.

The term “therapy” refers to the standard of care needed to treat a specific disease or disorder. In a typical example, therapy involves the act of administering to a subject a therapeutic agent(s) in an effective amount. For example, a therapeutic agent for treating a subject having or predicted to develop sepsis may include an antibiotic, which include, but are not limited to, penicillins, cephalosporins, fluroquinolones, tetracyclines, macrolides, and aminoglycosides. In some embodiments, treatment for sepsis may include hydration, including but not limited to normal saline, lactated ringers solution, or osmotic solutions such as albumin. Treatment for sepsis may also include transfusion of blood products or the administration of vasopressors including but not limited to norepinephrine, epinephrine, dopamine, vasopressin, or dobutamine. Some patients with sepsis will have respiratory failure and may require ventilator assistance including but not limited to biphasic positive airway pressure or intubation and ventilation. Other agents for treating sepsis include non-steroidal anti-inflammatory agents or anti-pyretic agents.

As used herein, the terms “treat”, “treatment” and “treating” refer to the reduction or amelioration of the severity, duration and/or progression of a disease or disorder such as sepsis, or one or more symptoms thereof resulting, from the administration of one or more therapies.

### Detailed Description of Certain Embodiments

In one aspect, the present disclosure provides a method of diagnosing and treating sepsis in a burn subject comprising, measuring one or more biomarkers in a first sample obtained from the burn subject, wherein the one or more biomarkers comprise one or a combination of expression products from the group of genes comprising ARG1A, ARG1B, ATG2A, BCL2A1, BMX, CD177, CEACAM4, CLEC4D, CLEC4D_A, HP, HPR, IL18R1, IL18RAP, MMP8, MS4A4A, PADI4, PFKFB2, PLAC8_A, RNASE2, SIGLEC5, STOM, TDRD9, VNN1, VNN1_2, or ZDHHC20; determining whether the burn subject has a probability of developing sepsis based on the measurement of the one or more biomarkers in the sample; and administering to the burn subject a sepsis therapy. It is noted that reference to ARG1A and ARG1B refer to the same gene ARG1, but the nomenclature of ARG1A and ARG1B is used to denote the two different transcripts produced by ARG1. Similarly, CLEC4D and CLEC4D_A refer to the same gene, CLEC4D but produces different transcripts CLEC4D and CLEC4D_A. Similarly, VNN1 and VNN1_2 refer to the same gene VNN1 that produces these two transcripts. PLAC8_A refers to a transcript of gene PLAC8.

In certain embodiments, methods of predicting sepsis in a burn patient are developed based on the transcriptomics data derived from sepsis patients.

Also provided are the mathematical operations needed to assess the risk based on the measurements of a set of molecules (such as transcriptome, epigenome, proteome, metabolome and so on). In the logistic regression model described by this algorithm,

$$logit ( P ) = a + {bX}_{1} + {cX}_{2} + … + {nX}_{n} \qquad ( Equation 1 )$$

where logit( ) is the log odds function of a value, P is the probability of developing illness (such as sepsis, and so on), a is the intercept of the equation, b through n are coefficient estimates of the independent variables, and X1 through Xn are the expression values of the molecules used as independent variables in this model.

To apply this algorithm, the user must multiply the molecular status (such as regulation, fold change, abundance and so on) by their corresponding coefficient described in the algorithm, sum the products, and add the intercept a described by the algorithm to the summed products. The resulting value is the log of the odds of developing illness (such as sepsis, sleep deprivation and so on).

The molecular input and the numerical figures (regulations and coefficients) are provided in Tables 2A and 3A. Tables 2A and 3A list differentially expressed genes (i.e., gene expression between burn patients who experienced sepsis and burn patients who did not experience sepsis) by the gene names, their regulations (derived from dual dye cDNA microarray of whole genome analysis) and corresponding coefficients (b, c, . . . n from Equation 1).

In some embodiments, the measuring one or more biomarkers in a sample comprises a clinical assessment or a molecular assessment. In some embodiments, the clinical assessment comprises a physiological measurement, a biometric measurement, a psychological measurement, or a clinical lab assay. In some embodiments, the molecular assessment comprises a nucleic acid sequencing assay, a next generation nucleic acid sequencing, (NGS) assay, a Sanger sequencing assay, a PCR assay, a quantitative PCR (qPCR) assay, a reverse transcription PCR (RT-PCR) assay, a miRNA assay, a microarray assay, a Northern blot assay, a Southern blot assay, a luciferase assay, a fluorescence immunoassay, a radio immunoassay, an enzyme-linked immunosorbent assay (ELISA), a flow cytometry assay, a mass spectrometry (MS) assay, a Selected Reaction Monitoring (SRM-MS) assay, a Sequential Windowed data independent Acquisition of the Total High resolution Mass Spectroscopy (SWATH-MS) assay, a Western blot assay, a genome wide methylation assay, a targeted methylation assay, a bisulfite methylation sequencing assay, a restriction enzyme methylation sequencing assay, a high performance liquid chromatography (HPLC) assay, an ultrahigh performance liquid chromatography (UHPLC) assay, a mass spectrometry (MS) assay, an ultrahigh performance liquid chromatography/tandem mass spectrometry (UHPLC/MS/MS2), a gas chromatography/mass spectrometry (GC/MS) assay, a lipidomics assay, a cell aging assay, an endocrine assay, a neuroendocrine assay, a cytokine assay, or an immune cell assay. In a specific embodiment measuring one or more biomarkers involves qPCR using select probes for detection of select genes, e.g., one or more of the probes outlined in Table 1A and SEQ ID NOs 1-25.

In other embodiments, provided is a machine learning system that generates a predictive model that may be static. In other words, the predictive model is trained and then its use is implemented with a computer implemented system wherein data values (e.g. biomarker marker measurements and age) are inputted and the predictive model provides an output that is used to discern burn subjects at risk of developing sepsis.

In other embodiments, the predictive models are continuously, or routinely, being updated and improved wherein the input values, output values, along with a diagnostic indicator from patients are used to further train the classifier models. In embodiments, the classifier model has an improved performance of a Receiver Operator Characteristic (ROC) curve having a sensitivity value of at least 0.8 and a specificity value of at least 0.65.

In embodiments, the predictive model is further trained and improved by the machine learning system comprising (1) obtaining one or more test results from the diagnostic testing which confirm or deny the presence of sepsis in the burn patient, (2) incorporating the one or more test results into the training data for further training of the predictive model of the machine learning system; and (3) generating an improved predictive model by the machine learning system.

In embodiments provided herein is a predictive model to predict an increased risk of developing sepsis in a burn patient. In embodiments, this first predictive model is generated by a machine learning system using training data that comprises values of a panel of at least two biomarkers, age, and a diagnostic indicator, for a population of patients. In certain embodiments, the training data comprises values of a panel of at least 2-6 biomarkers. In embodiments, the training data comprises values from a panel of biomarkers set forth in Tables 1A and 1B and SEQ ID NOs 1-50.

### Fragments and Variants of Biomarkers

Also contemplated herein is the detection of fragments or variants of a biomarker disclosed herein for predicting risk or probability of burn patients to develop sepsis. Fragments of a transcript of a gene can include a portion of the full gene transcript. In certain embodiments the fragment comprises 10-2000 contiguous bases of the full gene transcript.

In certain embodiments, a gene or transcript thereof may possess variability from individual to individual or within the biological milieu of a subject. Variants of a gene or gene transcript are typically those that possess a defined level of sequence identity.

Generally, variants of a particular biomarker gene or polynucleotide will have at least about 40%, 45%, 50%, 51%, 52%, 53%, 54%, 55%, 56%, 57%, 58%, 59% 60%, 61%, 62%, 63%, 64%, 65%, 66%, 67%, 68%, 69% 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more sequence identity to that particular nucleotide sequence as determined by sequence alignment programs known in the art using default parameters. In some embodiments, the Biomarker gene or polynucleotide displays at least about 40%, 45%, 50%, 51%, 52%, 53%, 54%, 55%, 56%, 57%, 58%, 59% 60%, 61%, 62%, 63%, 64%, 65%, 66%, 67%, 68%, 69% 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a nucleotide sequence of ARG1A, ARG1B, ATG2A, BCL2A1, BMX, CD177, CEACAM4, CLEC4D, CLEC4D_A, HP, HPR, IL18R1, IL18RAP, MMP8, MS4A4A, PADI4, PFKFB2, PLAC8_A, RNASE2, SIGLEC5, STOM, TDRD9, VNN1, VNN1_2, or ZDHHC20, or a sequence selected from any one of SEQ ID NO: 1-25 or 26-50.

Corresponding Biomarkers also include amino acid sequence that displays substantial sequence similarity or identity to the amino acid sequence of a reference Biomarker polypeptide. In general, an amino acid sequence that corresponds to a reference amino acid sequence will display at least about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 97, 88, 89, 90, 91, 92, 93, 94, 95, 96, 97, 98, 99% or even up to 100% sequence similarity or identity to a reference amino acid sequence.

In some embodiments, calculations of sequence similarity or sequence identity between sequences are performed as follows:

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In some embodiments, the length of a reference sequence aligned for comparison purposes is at least 30%, usually at least 40%, more usually at least 50%, 60%, and even more usually at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide at the corresponding position in the second sequence, then the molecules are identical at that position. For amino acid sequence comparison, when a position in the first sequence is occupied by the same or similar amino acid residue (i.e., conservative substitution) at the corresponding position in the second sequence, then the molecules are similar at that position.

The percent identity between the two sequences is a function of the number of identical amino acid residues shared by the sequences at individual positions, taking into account the number of gaps, and the length of each gap, which need to be introduced for optimal alignment of the two sequences. By contrast, the percent similarity between the two sequences is a function of the number of identical and similar amino acid residues shared by the sequences at individual positions, taking into account the number of gaps, and the length of each gap, which need to be introduced for optimal alignment of the two sequences.

The comparison of sequences and determination of percent identity or percent similarity between sequences can be accomplished using a mathematical algorithm. In certain embodiments, the percent identity or similarity between amino acid sequences is determined using the Needleman and Wunsch, (1970. J. Mol. Biol. 48:444-453) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. In specific embodiments, the percent identity between nucleotide sequences is determined using the GAP program in the GCG software package (available at www.gcg.com), using a NWSgapdna.CMP matrix and a gap weight of 40, 50, 60, 70, or 80 and a length weight of 1, 2, 3, 4, 5, or 6. An non-limiting set of parameters (and the one that should be used unless otherwise specified) includes a Blossum 62 scoring matrix with a gap penalty of 12, a gap extend penalty of 4, and a frameshift gap penalty of 5.

In some embodiments, the percent identity or similarity between amino acid or nucleotide sequences can be determined using the algorithm of E. Meyers and W. Miller (1989. Cabios. 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al., (1990, J. Mol. Biol, 215:403-10). BLAST nucleotide searches can be performed with the NBLAST program, score=100, wordlength=12 to obtain nucleotide sequences homologous to 53010 nucleic acid molecules of the invention. BLAST protein searches can be performed with the XBLAST program, score=50, wordlength=3 to obtain amino acid sequences homologous to 53010 protein molecules of the invention. To obtain gapped alignments for comparison purposes, Gapped BLAST can be utilized as described in Altschul et al., (1997, Nucleic Acids Res, 25:3389-3402). When utilizing BLAST and Gapped BLAST programs, the default parameters of the respective programs (e.g., XBLAST and NBLAST) can be used.

Corresponding Biomarker polynucleotides also include nucleic acid sequences that hybridize to reference Biomarker polynucleotides, or to their complements, under stringency conditions described below. As used herein, the term “hybridizes under low stringency, medium stringency, high stringency, or very high stringency conditions” describes conditions for hybridization and washing. “Hybridization” is used herein to denote the pairing of complementary nucleotide sequences to produce a DNA-DNA hybrid or a DNA-RNA hybrid. Complementary base sequences are those sequences that are related by the base-pairing rules. In DNA, A pairs with T and C pairs with G. In RNA, U pairs with A and C pairs with G. In this regard, the terms “match” and “mismatch” as used herein refer to the hybridization potential of paired nucleotides in complementary nucleic acid strands. Matched nucleotides hybridize efficiently, such as the classical A-T and G-C base pair mentioned above. Mismatches are other combinations of nucleotides that do not hybridize efficiently.

Guidance for performing hybridization reactions can be found in Ausubel et al., (1998, supra), Sections 6.3.1-6.3.6. Aqueous and non-aqueous methods are described in that reference and either can be used. Reference herein to low stringency conditions include and encompass from at least about 1% v/v to at least about 15% v/v formamide and from at least about 1 M to at least about 2 M salt for hybridization at 42° C., and at least about 1 M to at least about 2 M salt for washing at 42° C. Low stringency conditions also may include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at room temperature. One embodiment of low stringency conditions includes hybridization in 6× sodium chloride/sodium citrate (SSC) at about 45□□C, followed by two washes in 0.2×SSC, 0.1% SDS at least at 50° C. (the temperature of the washes can be increased to 55° C. for low stringency conditions). Medium stringency conditions include and encompass from at least about 16% v/v to at least about 30% v/v formamide and from at least about 0.5 M to at least about 0.9 M salt for hybridization at 42° C., and at least about 0.1 M to at least about 0.2 M salt for washing at 55° C. Medium stringency conditions also may include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at 60-65° C. One embodiment of medium stringency conditions includes hybridizing in 6×SSC at about 45□□C, followed by one or more washes in 0.2×SSC, 0.1% SDS at 60° C. High stringency conditions include and encompass from at least about 31% v/v to at least about 50% v/v formamide and from about 0.01 M to about 0.15 M salt for hybridization at 42° C., and about 0.01 M to about 0.02 M salt for washing at 55° C. High stringency conditions also may include 1% BSA, 1 mM EDTA, 0.5 M NaHPO₄ (pH 7.2), SDS for hybridization at 65° C., and (i) 0.2×SSC, 7% 0.1% SDS; or (ii) 0.5% BSA, 1 mM EDTA, 40 mM NaHPO₄ (pH 7.2), 1% SDS for washing at a temperature in excess of 65° C. One embodiment of high stringency conditions includes hybridizing in 6×SSC at about 45□□C, followed by one or more washes in 0.2×SSC, 0.1% SDS at 65° C.

In certain embodiments, a corresponding Biomarker polynucleotide is one that hybridizes to a disclosed nucleotide sequence under very high stringency conditions. One embodiment of very high stringency conditions includes hybridizing 0.5 M sodium phosphate, 7% SDS at 65° C., followed by one or more washes at 0.2×SSC, 1% SDS at 65° C.

Other stringency conditions are well known in the art and a skilled addressee will recognize that various factors can be manipulated to optimize the specificity of the hybridization. Optimization of the stringency of the final washes can serve to ensure a high degree of hybridization. For detailed examples, see Ausubel et al., supra at pages 2.10.1 to 2.10.16 and Sambrook et al. (1989, supra) at sections 1.101 to 1.104.

### Detection and Measurement of Biomarkers

In some embodiments, detecting comprises an instrument, i.e., using an automated or semi-automated detecting means that can, but needs not, comprise a computer algorithm. In some embodiments, the instrument is portable, transportable or comprises a portable component which can be inserted into a less mobile or transportable component, e.g., residing in a laboratory, hospital or other environment in which detection of amplification products is conducted. In certain embodiments, the detecting step is combined with or is a continuation of at least one amplification step, one sequencing step, one isolation step, one separating step, for example but not limited to a capillary electrophoresis instrument comprising at least one fluorescent scanner and at least one graphing, recording, or readout component; a chromatography column coupled with an absorbance monitor or fluorescence scanner and a graph recorder; a chromatography column coupled with a mass spectrometer comprising a recording and/or a detection component; a spectrophotometer instrument comprising at least one UV/visible light scanner and at least one graphing, recording, or readout component; a microarray with a data recording device such as a scanner or CCD camera; or a sequencing instrument with detection components selected from a sequencing instrument comprising at least one fluorescent scanner and at least one graphing, recording, or readout component, a sequencing by synthesis instrument comprising fluorophore-labeled, reversible-terminator nucleotides, a pyro sequencing method comprising detection of pyrophosphate (PPi) release following incorporation of a nucleotide by DNA polymerase, pair-end sequencing, polony sequencing, single molecule sequencing, nanopore sequencing, and sequencing by hybridization or by ligation as discussed in Lin, B. et al. “Recent Patents on Biomedical Engineering (2008) 1 (1) 60-67, incorporated by reference herein.

In certain embodiments, the detecting step is combined with an amplifying step, for example but not limited to, real-time analysis such as Q-PCR. Exemplary means for performing a detecting step include the ABI PRISM® Genetic Analyzer instrument series, the ABI PRISM® DNA Analyzer instrument series, the ABI PRISM® Sequence Detection Systems instrument series, and the Applied Biosystems Real-Time PCR instrument series (all from Applied Biosystems); and microarrays and related software such as the Applied Biosystems microarray and Applied Biosystems 1700 Chemiluminescent Microarray Analyzer and other commercially available microarray and analysis systems available from Affymetrix, Agilent, and Amersham Biosciences, among others (see also Gerry et al., J. Mol. Biol. 292:251-62, 1999; De Bellis et al., Minerva Biotec 14:247-52, 2002; and Stears et al., Nat. Med. 9:140-45, including supplements, 2003) or bead array platforms (Illumina, San Diego, Calif.). Exemplary software includes GeneMapper™ Software, GeneScan® Analysis Software, and Genotyper® software (all from Applied Biosystems).

In some embodiments, an amplification product can be detected and quantified based on the mass-to-charge ratio of at least a part of the amplicon (m/z). For example, in some embodiments, a primer comprises a mass spectrometry-compatible reporter group, including without limitation, mass tags, charge tags, cleavable portions, or isotopes that are incorporated into an amplification product and can be used for mass spectrometer detection (see, e.g., Haff and Smirnov, Nucl. Acids Res. 25:3749-50, 1997; and Sauer et al., Nucl. Acids Res. 31: e63, 2003). An amplification product can be detected by mass spectrometry. In some embodiments, a primer comprises a restriction enzyme site, a cleavable portion, or the like, to facilitate release of a part of an amplification product for detection. In certain embodiments, a multiplicity of amplification products are separated by liquid chromatography or capillary electrophoresis, subjected to ESI or to MALDI, and detected by mass spectrometry. Descriptions of mass spectrometry can be found in, among other places, The Expanding Role of Mass Spectrometry in Biotechnology, Gary Siuzdak, MCC Press, 2003.

In some embodiments, detecting comprises a manual or visual readout or evaluation, or combinations thereof. In some embodiments, detecting comprises an automated or semi-automated digital or analog readout. In some embodiments, detecting comprises real-time or endpoint analysis. In some embodiments, detecting comprises a microfluidic device, including without limitation, a TaqMan® Low Density Array (Applied Biosystems). In some embodiments, detecting comprises a real-time detection instrument. Exemplary real-time instruments include, the ABI PRISM® 7000 Sequence Detection System, the ABI PRISM® 7700 Sequence Detection System, the Applied Biosystems 7300 Real-Time PCR System, the Applied Biosystems 7500 Real-Time PCR System, the Applied Biosystems 7900 HT Fast Real-Time PCR System (all from Applied Biosystems); the LightCycler™ System (Roche Molecular); the Mx3000P™ Real-Time PCR System, the Mx3005PT Real-Time PCR System, and the Mx4000® Multiplex Quantitative PCR System (Stratagene, La Jolla, Calif.); and the Smart Cycler System (Cepheid, distributed by Fisher Scientific). Descriptions of real-time instruments can be found in, among other places, their respective manufacturer's user's manuals; McPherson; DNA Amplification: Current Technologies and Applications, Demidov and Broude, eds., Horizon Bioscience, 2004; and U.S. Pat. No. 6,814,934.

The term “amplification reaction mixture” and/or “master mix” may refer to an aqueous solution comprising the various (some or all) reagents used to amplify a target nucleic acid. Such reactions may also be performed using solid supports or semi-solid supports (e.g., an array). The reactions may also be performed in single or multiplex format as desired by the user. These reactions typically include enzymes, aqueous buffers, salts, amplification primers, target nucleic acid, and nucleoside triphosphates. In some embodiments, the amplification reaction mix and/or master mix may include one or more of, for example, a buffer (e.g., Tris), one or more salts (e.g., MgC, KCl), glycerol, dNTPs (dA, dT, dG, dC, dU), recombinant BSA (bovine serum albumin), a dye (e.g., ROX passive reference dye), one or more detergents, polyethylene glycol (PEG), polyvinyl pyrrolidone (PVP), gelatin (e.g., fish or bovine source) and/or antifoam agent. Depending upon the context, the mixture can be either a complete or incomplete amplification reaction mixture. In some embodiments, the master mix does not include amplification primers prior to use in an amplification reaction. In some embodiments, the master mix does not include target nucleic acid prior to use in an amplification reaction. In some embodiments, an amplification master mix is mixed with a target nucleic acid sample prior to contact with amplification primers.

In some embodiments, the amplification reaction mixture comprises amplification primers and a master mix. In some embodiments, the amplification reaction mixture comprises amplification primers, a probe (e.g. detectably labeled probe), and a master mix. In a specific embodiment, the probe comprises a sequence selected from SEQ ID NOs 1-25.

In some embodiments, the reaction mixture of amplification primers and master mix or amplification primers, probe and master mix are dried in a storage vessel or reaction vessel. In some embodiments, the reaction mixture of amplification primers and master mix or amplification primers, probe and master mix are lyophilized in a storage vessel or reaction vessel. In some embodiments, the disclosure generally relates to the amplification of multiple target-specific sequences from a single control nucleic acid molecule. For example, in some embodiments that single control nucleic acid molecule can include RNA and in other embodiments, that single control nucleic acid molecule can include DNA. In some embodiments, the target-specific primers and primer pairs are target-specific sequences that can amplify specific regions of a nucleic acid molecule, for example, a control nucleic acid molecule. In some embodiments, the target-specific primers can prime reverse transcription of RNA to generate target-specific cDNA. In some embodiments, the target-specific primers can amplify target DNA or cDNA. In some embodiments, the amount of DNA required for selective amplification can be from about 1 ng to 1 microgram. In some embodiments, the amount of DNA required for selective amplification of one or more target sequences can be about 1 ng, about 5 ng or about 10 ng. In some embodiments, the amount of DNA required for selective amplification of target sequence is about 10 ng to about 200 ng.

As used herein, the term “reaction vessel” generally refers to any container, chamber, device, or assembly, in which a reaction can occur in accordance with the present teachings. In some embodiments, a reaction vessel may be a microtube, for example, but not limited to, a 0.2 mL or a 0.5 mL reaction tube such as a Micro Amp™ Optical tube (Life Technologies Corp., Carlsbad, Calif.) or a micro-centrifuge tube, or other containers of the sort in common practice in molecular biology laboratories. In some embodiments, a reaction vessel comprises a well of a multi-well plate (such as a 48-, 96-, or 384-well microtiter plate), a spot on a glass slide, a well in a TaqMan™ Array Card or a channel or chamber of a microfluidics device, including without limitation a TaqMan™ Low Density Array, or a through-hole of a TaqMan™ OpenArray™ Real-Time PCR plate (Applied Biosystems, Thermo Fisher Scientific). For example, but not as a limitation, a plurality of reaction vessels can reside on the same support. An OpenArray™ Plate, for example, is a reaction plate 3072 through-holes. Each such through-hole in such a plate may contain a single TaqMan™ assay. In some embodiments, lab-on-a-chip-like devices available, for example, from Caliper or Fluidigm can provide reaction vessels. It will be recognized that a variety of reaction vessels are commercially available or can be designed for use in the context of the present teachings.

The terms “annealing” and “hybridizing”, including, without limitation, variations of the root words “hybridize” and “anneal”, are used interchangeably and mean the nucleotide base—pairing interaction of one nucleic acid with another nucleic acid that results in the formation of a duplex, triplex, or other higher-ordered structure. The primary interaction is typically nucleotide base specific, e.g., A:T, A:U, and G:C, by Watson-Crick and Hoogsteen-type hydrogen bonding. In certain embodiments, base-stacking and hydrophobic interactions may also contribute to duplex stability. Conditions under which primers and probes anneal to complementary sequences are well known in the art, e.g., as described in Nucleic Acid Hybridization, A Practical Approach, Hames and Higgins, eds., IRL Press, Washington, D.C. (1985) and Wetmur and Davidson, Mol. Biol. 31:349 (1968).

In general, whether such annealing takes place is influenced by, among other things, the length of the complementary portions of the complementary portions of the primers and their corresponding binding sites in the target flanking sequences and/or amplicons, or the corresponding complementary portions of a reporter probe and its binding site; the pH; the temperature; the presence of mono- and divalent cations; the proportion of G and C nucleotides in the hybridizing region; the viscosity of the medium; and the presence of denaturants. Such variables influence the time required for hybridization. Thus, the preferred annealing conditions will depend upon the particular application. Such conditions, however, can be routinely determined by persons of ordinary skill in the art, without undue experimentation. Preferably, annealing conditions are selected to allow the primers and/or probes to selectively hybridize with a complementary sequence in the corresponding target flanking sequence or amplicon, but not hybridize to any significant degree to different target nucleic acids or non-target sequences in the reaction composition at the second reaction temperature.

### Illustrated System and Method Embodiments

FIG. 1 is a block diagram that illustrates an example of a system 100 for predicting whether a burn patient will experience sepsis, according to one embodiment. As illustrated in FIG. 1A, a system 100 includes a biomarker measurement device 102 configured to measure data that indicates values for one or more biomarkers a burn patient. In one example, the biomarker measurement device 102 is a device that measures gene transcript levels of selected biomarker genes. The device 102 is typically one that can amplify/copy a target amplicon and quantify the number of copies/amplicons generated herein. The amplification process could be temperature controlled or not. The amplicon could be a template based on DNA, RNA, cDNA. In a specific embodiment, the biomarker measurement devices is a PCR machine.

As further illustrated in FIG. 1, the system 100 includes a data processing system 104 connected to the biomarker measurement device 102, to receive the data of the values of the one or more biomarkers. The data processing system 104 includes a process 112 to predict whether the patient will require a sepsis therapy. In some embodiments, the data processing system 104 is a computer system as described below with reference to FIG. 4 or a chip set described below with reference to FIG. 5. The process 112 is configured to cause the system 100 to apply coefficients to the values of the one or more biomarkers and to determine second data that indicates a prediction that the patient will require sepsis therapy based on applying the coefficients to the values of the one or more biomarkers. The hardware used to form the data processing system 104 of the system 100 is described in more detail below in the Hardware Overview section.

In addition to the biomarker values of the one or more biomarkers, the data processing system 104 may receive third data that indicates values for one or more secondary parameters of a characteristic of the patient, such as an age and a gender of the patient, for example. FIG. 1A illustrates that the system 100 may include a manual input 108 such as a keyboard or a touchscreen, for example, to manually enter the values of the one or more biomarkers, age and/or gender, or other physiological characteristics of the burn patient. Alternatively, FIG. 1A illustrates the system 100 may include a patient database 110 connected to the data processing system 104 that includes collected data from past burn patients for further refinement of the coefficients to be applied to values of one or more biomarkers.

FIG. 2 is a flow diagram that illustrates an example of a method 200 for predicting that a burn subject will experience sepsis, according to one embodiment. Although the flow diagram of FIG. 2, and subsequent flow diagram FIG. 3A, is each depicted as integral steps in a particular order for purposes of illustration, in other embodiments one or more steps, or portions thereof, are performed in a different order, or overlapping in time, in series or in parallel, or are deleted, or one or more other steps are added, or the method is changed in some combination of ways.

After starting at block 201, in step 202, data is obtained, on the data processing system 104, pertaining to values for one or more biomarkers in a sample of the burn subject. In step 204, coefficients are applied, on the data processing system 104, to the values for the one or more biomarker values. In step 206, a prediction is determined, on the data processing system 104, that the burn subject will experience sepsis. In step 208, a determination is made, on the data processing system 104, on whether to administer a sepsis therapy, based on the prediction, before the method ends at block 209.

In one embodiment, the biomarker values of the one or more biomarkers are expression values for one or more expression products of genes selected from the group of genes comprising ARG1A, ARG1B, ATG2A, BCL2A1, BMX, CD177, CEACAM4, CLEC4D, CLEC4D_A, HP, HPR, IL18R1, IL18RAP, MMP8, MS4A4A, PADI4, PFKFB2, PLAC8_A, RNASE2, SIGLEC5, STOM, TDRD9, VNN1, VNN1_2, or ZDHHC20. Table 1A illustrates an example of values of these genes to which the coefficients are applied in step 204.

FIG. 3 a block diagram that illustrates an example of a method 300 for determining a model for predicting whether a burn patient will experience sepsis, according to one embodiment. After starting at block 301, in step 302, data is obtained, on the data processing system 104, that indicates values for one or more biomarkers. In step 304, a result is assigned, on the data processing system 104, for each patient based on whether the burn patient experienced sepsis. In step 306, the data is fitted, on the data processing system 104, to the results for the plurality of patients. In step 308, the coefficients are determined, on the data processing system 104, for the one or more biomarkers, to determine a model for predicting whether a patient will experience sepsis based on an input of the one or more biomarkers, before the method ends at block 309.

### System Hardware

FIG. 4 is a block diagram that illustrates a computer system 400 upon which an embodiment of the invention may be implemented. Computer system 400 includes a communication mechanism such as a bus 410 for passing information between other internal and external components of the computer system 400. Information is represented as physical signals of a measurable phenomenon, typically electric voltages, but including, in other embodiments, such phenomena as magnetic, electromagnetic, pressure, chemical, molecular atomic and quantum interactions. For example, north and south magnetic fields, or a zero and non-zero electric voltage, represent two states (0, 1) of a binary digit (bit).). Other phenomena can represent digits of a higher base. A superposition of multiple simultaneous quantum states before measurement represents a quantum bit (qubit). A sequence of one or more digits constitutes digital data that is used to represent a number or code for a character. In some embodiments, information called analog data is represented by a near continuum of measurable values within a particular range. Computer system 400, or a portion thereof, constitutes a means for performing one or more steps of one or more methods described herein.

A sequence of binary digits constitutes digital data that is used to represent a number or code for a character. A bus 410 includes many parallel conductors of information so that information is transferred quickly among devices coupled to the bus 410. One or more processors 402 for processing information are coupled with the bus 410. A processor 402 performs a set of operations on information. The set of operations include bringing information in from the bus 410 and placing information on the bus 410. The set of operations also typically include comparing two or more units of information, shifting positions of units of information, and combining two or more units of information, such as by addition or multiplication. A sequence of operations to be executed by the processor 402 constitutes computer instructions.

Computer system 400 also includes a memory 404 coupled to bus 410. The memory 404, such as a random access memory (RAM) or other dynamic storage device, stores information including computer instructions. Dynamic memory allows information stored therein to be changed by the computer system 400. RAM allows a unit of information stored at a location called a memory address to be stored and retrieved independently of information at neighboring addresses. The memory 404 is also used by the processor 402 to store temporary values during execution of computer instructions. The computer system 400 also includes a read only memory (ROM) 406 or other static storage device coupled to the bus 410 for storing static information, including instructions, that is not changed by the computer system 400. Also coupled to bus 410 is a non-volatile (persistent) storage device 408, such as a magnetic disk or optical disk, for storing information, including instructions, that persists even when the computer system 400 is turned off or otherwise loses power.

Information, including instructions, is provided to the bus 410 for use by the processor from an external input device 412, such as a keyboard containing alphanumeric keys operated by a human user, or a sensor. A sensor detects conditions in its vicinity and transforms those detections into signals compatible with the signals used to represent information in computer system 400. Other external devices coupled to bus 410, used primarily for interacting with humans, include a display device 414, such as a cathode ray tube (CRT) or a liquid crystal display (LCD), for presenting images, and a pointing device 416, such as a mouse or a trackball or cursor direction keys, for controlling a position of a small cursor image presented on the display 414 and issuing commands associated with graphical elements presented on the display 414.

In the illustrated embodiment, special purpose hardware, such as an application specific integrated circuit (IC) 420, is coupled to bus 410. The special purpose hardware is configured to perform operations not performed by processor 402 quickly enough for special purposes. Examples of application specific ICs include graphics accelerator cards for generating images for display 414, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 400 also includes one or more instances of a communications interface 470 coupled to bus 410. Communication interface 470 provides a two-way communication coupling to a variety of external devices that operate with their own processors, such as printers, scanners and external disks. In general the coupling is with a network link 478 that is connected to a local network 480 to which a variety of external devices with their own processors are connected. For example, communication interface 470 may be a parallel port or a serial port or a universal serial bus (USB) port on a personal computer. In some embodiments, communications interface 470 is an integrated services digital network (ISDN) card or a digital subscriber line (DSL) card or a telephone modem that provides an information communication connection to a corresponding type of telephone line. In some embodiments, a communication interface 470 is a cable modem that converts signals on bus 410 into signals for a communication connection over a coaxial cable or into optical signals for a communication connection over a fiber optic cable. As another example, communications interface 470 may be a local area network (LAN) card to provide a data communication connection to a compatible LAN, such as Ethernet. Wireless links may also be implemented. Carrier waves, such as acoustic waves and electromagnetic waves, including radio, optical and infrared waves travel through space without wires or cables. Signals include man-made variations in amplitude, frequency, phase, polarization or other physical properties of carrier waves. For wireless links, the communications interface 470 sends and receives electrical, acoustic or electromagnetic signals, including infrared and optical signals that carry information streams, such as digital data.

The term computer-readable medium is used herein to refer to any medium that participates in providing information to processor 402, including instructions for execution. Such a medium may take many forms, including, but not limited to, non-volatile media, volatile media and transmission media. Non-volatile media include, for example, optical or magnetic disks, such as storage device 408. Volatile media include, for example, dynamic memory 404. Transmission media include, for example, coaxial cables, copper wire, fiber optic cables, and waves that travel through space without wires or cables, such as acoustic waves and electromagnetic waves, including radio, optical and infrared waves. The term computer-readable storage medium is used herein to refer to any medium that participates in providing information to processor 402, except for transmission media.

Common forms of computer-readable media include, for example, a floppy disk, a flexible disk, a hard disk, a magnetic tape, or any other magnetic medium, a compact disk ROM (CD-ROM), a digital video disk (DVD) or any other optical medium, punch cards, paper tape, or any other physical medium with patterns of holes, a RAM, a programmable ROM (PROM), an erasable PROM (EPROM), a FLASH-EPROM, or any other memory chip or cartridge, a carrier wave, or any other medium from which a computer can read. The term non-transitory computer-readable storage medium is used herein to refer to any medium that participates in providing information to processor 402, except for carrier waves and other signals.

Logic encoded in one or more tangible media includes one or both of processor instructions on a computer-readable storage media and special purpose hardware, such as ASIC 420.

Network link 478 typically provides information communication through one or more networks to other devices that use or process the information. For example, network link 478 may provide a connection through local network 480 to a host computer 482 or to equipment 484 operated by an Internet Service Provider (ISP). ISP equipment 484 in turn provides data communication services through the public, world-wide packet-switching communication network of networks now commonly referred to as the Internet 490. A computer called a server 492 connected to the Internet provides a service in response to information received over the Internet. For example, server 492 provides information representing video data for presentation at display 414.

The invention is related to the use of computer system 400 for implementing the techniques described herein. According to one embodiment of the invention, those techniques are performed by computer system 400 in response to processor 402 executing one or more sequences of one or more instructions contained in memory 404. Such instructions, also called software and program code, may be read into memory 404 from another computer-readable medium such as storage device 408. Execution of the sequences of instructions contained in memory 404 causes processor 402 to perform the method steps described herein. In alternative embodiments, hardware, such as application specific integrated circuit 420, may be used in place of or in combination with software to implement the invention. Thus, embodiments of the invention are not limited to any specific combination of hardware and software.

The signals transmitted over network link 478 and other networks through communications interface 470, carry information to and from computer system 400. Computer system 400 can send and receive information, including program code, through the networks 480, 490 among others, through network link 478 and communications interface 470. In an example using the Internet 490, a server 492 transmits program code for a particular application, requested by a message sent from computer 400, through Internet 490, ISP equipment 484, local network 480 and communications interface 470. The received code may be executed by processor 402 as it is received, or may be stored in storage device 408 or other non-volatile storage for later execution, or both. In this manner, computer system 400 may obtain application program code in the form of a signal on a carrier wave.

Various forms of computer readable media may be involved in carrying one or more sequence of instructions or data or both to processor 402 for execution. For example, instructions and data may initially be carried on a magnetic disk of a remote computer such as host 482. The remote computer loads the instructions and data into its dynamic memory and sends the instructions and data over a telephone line using a modem. A modem local to the computer system 400 receives the instructions and data on a telephone line and uses an infra-red transmitter to convert the instructions and data to a signal on an infra-red a carrier wave serving as the network link 478. An infrared detector serving as communications interface 470 receives the instructions and data carried in the infrared signal and places information representing the instructions and data onto bus 410. Bus 410 carries the information to memory 404 from which processor 402 retrieves and executes the instructions using some of the data sent with the instructions. The instructions and data received in memory 404 may optionally be stored on storage device 408, either before or after execution by the processor 402.

FIG. 5 illustrates a chip set 500 upon which an embodiment of the invention may be implemented. Chip set 500 is programmed to perform one or more steps of a method described herein and includes, for instance, the processor and memory components described with respect to FIG. 4 incorporated in one or more physical packages (e.g., chips). By way of example, a physical package includes an arrangement of one or more materials, components, and/or wires on a structural assembly (e.g., a baseboard) to provide one or more characteristics such as physical strength, conservation of size, and/or limitation of electrical interaction. It is contemplated that in certain embodiments the chip set can be implemented in a single chip. Chip set 500, or a portion thereof, constitutes a means for performing one or more steps of a method described herein.

In one embodiment, the chip set 500 includes a communication mechanism such as a bus 501 for passing information among the components of the chip set 500. A processor 503 has connectivity to the bus 501 to execute instructions and process information stored in, for example, a memory 505. The processor 503 may include one or more processing cores with each core configured to perform independently. A multi-core processor enables multiprocessing within a single physical package. Examples of a multi-core processor include two, four, eight, or greater numbers of processing cores.

Alternatively or in addition, the processor 503 may include one or more microprocessors configured in tandem via the bus 501 to enable independent execution of instructions, pipelining, and multithreading. The processor 503 may also be accompanied with one or more specialized components to perform certain processing functions and tasks such as one or more digital signal processors (DSP) 507, or one or more application-specific integrated circuits (ASIC) 509. A DSP 507 typically is configured to process real-world signals (e.g., sound) in real time independently of the processor 503. Similarly, an ASIC 509 can be configured to performed specialized functions not easily performed by a general purposed processor. Other specialized components to aid in performing the inventive functions described herein include one or more field programmable gate arrays (FPGA) (not shown), one or more controllers (not shown), or one or more other special-purpose computer chips.

The processor 503 and accompanying components have connectivity to the memory 505 via the bus 501. The memory 505 includes both dynamic memory (e.g., RAM, magnetic disk, writable optical disk, etc.) and static memory (e.g., ROM, CD-ROM, etc.) for storing executable instructions that when executed perform one or more steps of a method described herein. The memory 505 also stores the data associated with or generated by the execution of one or more steps of the methods described herein.

### Kits

One or more biomarkers, one or more reagents for testing the biomarkers, sepsis risk factor parameters, a risk categorization table and/or system or software application capable of communicating with a machine learning system for determining a risk score, and any combinations thereof are amenable to the formation of kits (such as panels) for use in performing the present methods.

Compositions of the invention can include kits for prognosing whether a burn subject will develop sepsis. As used herein, “kit” or “kits” means any manufacture (e.g., a package or a container) including at least one reagent, such as a nucleic acid probe or the like, for specifically detecting the expression of the biomarkers described herein.

As used herein, “probe” means any molecule that is capable of selectively binding to a specifically intended target biomolecule, for example, a nucleotide transcript or a protein encoded by or corresponding to a biomarker. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies and organic molecules. The kit will, in some embodiments, include an instructional insert, or contain instructions for use on a label or other surface available for print on the product.

When making polynucleotides for use as probes to the biomarkers (e.g., hybridization probes or primer sets), one of skill in the art can be further guided by knowledge of redundancy in the genetic code as shown below in Table 1.

TABLE 4
Redundancy in Genetic Code.

| Residue | Triplet Codons Encoding the Residue |
|---|---|
| Ala (A) | GCU, GCC, GCA, GCG |
| Arg (R) | CGU, CGC, CGA, CGG, AGA, AGG |
| Asn (N) | AAU, AAC |
| Asp (D) | GAU, GAC |
| Cys (C) | UGU, UGC |
| Gin (Q) | CAA, CAG |
| Glu (E) | GAA, GAG |
| Gly (G) | GGU, GGC, GGA, GGG |
| His (H) | CAU, CAC |
| Iie (I) | AUU, AUC, AUA |
| Leu (L) | UUA, UUG, CUU, CUC, CUA, CUG |
| Lys (K) | AAA, AAG |
| Met (M) | AUG |
| Phe (F) | UUU, UUC |
| Pro (P) | CCU, CCC, CCA, CCG |
| Ser (S) | UCU, UCC, UCA, UCG, AGU, AGC |
| Thr (T) | ACU, ACC, ACA, ACG |
| Trp (W) | UGG |
| Tyr (Y) | UAU, UAC |
| Val (V) | GUU, GUC, GUA, GUG |
| START | AUG |
| STOP | UAG, UGA, UAA |

Methods of synthesizing polynucleotides are well known in the art, such as cloning and digestion of the appropriate sequences, as well as direct chemical synthesis (e.g., ink-jet deposition and electrochemical synthesis). Methods of cloning polynucleotides are described, for example, in Copeland et al. (2001) Nat. Rev. Genet. 2:769-779; Current Protocols in Molecular Biology (Ausubel et al. eds., John Wiley & Sons 1995); Molecular Cloning: A Laboratory Manual, 3rd ed. (Sambrook & Russell eds., Cold Spring Harbor Press 2001); and PCR Cloning Protocols, 2nd ed. (Chen & Janes eds., Humana Press 2002). Methods of direct chemical synthesis of polynucleotides include, but are not limited to, the phosphotriester methods of Reese (1978) Tetrahedron 34:3143-3179 and Narang et al. (1979) Methods Enzymol. 68:90-98; the phosphodiester method of Brown et al. (1979) Methods Enzymol. 68:109-151; the diethylphosphoramidate method of Beaucage et al. (1981) Tetrahedron Lett. 22:1859-1862; and the solid support methods of Fodor et al.

(1991) Science 251:767-773; Pease et al. (1994) Proc. Natl. Acad Sci. USA 91:5022-5026; and Singh-Gasson et al. (1999) Nature Biotechnol. 17:974-978; as well as U.S. Pat. No. 4,485,066. See also, Peattie (1979) Proc. Natl. Acad Sci. USA 76:1760-1764; as well as EP U.S. Pat. No. 1,721,908; Int'l Patent Application Publication Nos. WO 2004/022770 and WO 2005/082923; US Patent Application Publication Nos. 2009/0062521 and 2011/0092685; and U.S. Pat. Nos. 6,521,427; 6,818,395; 7,521,178 and 7,910,726.

The kits can be promoted, distributed or sold as units for performing the methods described below. Additionally, the kits can contain a package insert describing the kit and methods for its use. For example, the insert can include instructions for correlating the level of biomarker expression measured with a patient's likelihood of cancer recurrence, long-term survival, and the like, and select the most appropriate treatment option accordingly.

The kits therefore can be used for prognosing development of sepsis in burn patients with biomarkers at the nucleic acid level. Such kits are compatible with both manual and automated nucleic acid detection techniques (e.g., gene arrays, Northern blotting or Southern blotting). These kits can include a plurality of probes, for example, from 2 to 30 nucleic acid probes that specifically bind to distinct biomarkers, fragments or variants thereof. Alternatively, the kits can contain at least 2 probes, at least 3 probes, at least 4 probes, at least 5 probes, at least 6 probes, at least 7 probes, at least 8 probes, at least 9 probes, at least 10 probes, at least 11 probes, at least 12 probes, at least 13 probes, at least 14 probes, at least 15 probes, at least 16 probes, at least 17 probes, at least 18 probes, at least 19 probes, or at least 20 probes. In one example, the kits described herein used 2-6 probes including selected from SEQ ID NOs 1-25.

The reagents included in the kit for quantifying one or more regions of interest may include an adsorbent which binds and retains at least one region of interest contained in a panel, solid supports (such as beads) to be used in connection with said absorbents, one or more detectable labels, etc. The adsorbent can be any of numerous adsorbents used in analytical chemistry and immunochemistry, including metal chelates, cationic groups, anionic groups, hydrophobic groups, antigens and antibodies.

In certain embodiments, the kit comprises the necessary reagents to quantify at least one expression product from at least one gene selected from ARG1A, ARG1B, ATG2A, BCL2A1, BMX, CD177, CEACAM4, CLEC4D, CLEC4D_A, HP, HPR, IL18R1, IL18RAP, MMP8, MS4A4A, PADI4, PFKFB2, PLAC8_A, RNASE2, SIGLEC5, STOM, TDRD9, VNN1, VNN1_2, or ZDHHC20.

In some embodiments, the kit further comprises computer readable media for performing some or all of the operations described herein. The kit may further comprise an apparatus or system comprising one or more processors operable to receive the concentration values from the measurement of markers in a sample and configured to execute computer readable media instructions to determine a biomarker composite score, combine the biomarker composite score with other risk factors to generate a master composite score and compare the master composite score to a stratified cohort population comprising multiple risk categories (e.g. a master risk categorization table) to provide a risk score.

Any or all of the kit reagents can be provided within containers that protect them from the external environment, such as in sealed containers. Positive and/or negative controls can be included in the kits to validate the activity and correct usage of reagents employed in accordance with the invention. Controls can include samples, such as tissue sections, cells fixed on glass slides, RNA preparations from tissues or cell lines, and the like, known to be either positive or negative for the presence of at least five different biomarkers. The design and use of controls is standard and well within the routine capabilities of one of skill in the art.

### EXAMPLES

### Example 1: Prediction of Sepsis in Burn Subject

The discovery/pilot dataset consisted of 15 (culture proven) septic burn patients and age/gender matched 15 burn patients without sepsis. This prospective cohort is a subset of the human subject volunteers described elsewhere²². The whole blood samples were collected from the burn patients' admission to ICU (time 0) and at 2, 4, 8, and 12 hours, then every 12 or 24 hours for 7 days, and at hospital days 14 and 21. The longitudinally collected blood specimens along with the clinical data library that is built on every patient across their course of hospitalization (age, gender, vitals, transfusions, injury severity, infection, co-morbidities, etc.) presented a valuable resource for biomarker discovery.

A group of burn patients developed sepsis while at the ICU and their whole blood samples were assayed to identify early biomarkers for sepsis.

Transcriptomics assay: The transcriptomics assay was conducted using Whole Genome Human cDNA chip (Agilent, Inc.) or high throughput microarray. Differential gene expression analysis (burn patients, who eventually developed sepsis versus those, who never developed sepsis) found a large number of transcripts meeting FDR<0.05.

To select features (markers), the mean variance in normalized expression was calculated across time points in each sample. Probes with a mean variance >1.0 were selected as potential markers. In cases where a probe had a pairwise Pearson correlation >0.8 to another highly variant probe, one member of the pair was removed from the data set to eliminate redundant signal. This down-selection strategy resulted in a set of differentially expressed genes that were validated by real time polymerized chain reaction (RT-PCR) or quantitative PCR (qPCR). In certain examples, the biomarkers are expression products of genes identified are listed in Table 1B. The log fold change values of throughput microarray and qPCR data were correlated using Pearson algorithm and significantly correlated (p<0.05). Furthermore, we presented that data where throughput microarray and qPCR are showing similar regulations.

FIGS. 7-31 show the bar and whisker plots of the genes. The white box covers the interquartile region (from upper quartile to lower quartile), which was intercepted by a line marking the average value. The whisker covers the maximum to minimum ranges of the data. The left and right box-whisker represent the throughput microarray and qPCR data, respectively.

Tables 1A and 1B list the gene names or the early biomarkers of sepsis. The table includes their average long change values calculated by throughput microarray and qPCR tools, the Pearson correlation values (r-values) highlighting the association between throughput microarray and qPCR data. The probe sequence column lists the sequences of the gene that we identified to be linked to sepsis risk.

In addition to the twenty five (25) early biomarkers of sepsis, the algorithm was formulated. The gene expressions and the algorithm together are predictive of sepsis onset in a burn subject within 24h of ICU admission. The algorithm using these 25 gene transcripts is displayed in FIG. 6.

Towards the goal, two processes named K-fold cross validation and Random Single Bin Multiple Repeats (RSBMR) were used to find best fitting predictive models. For both processes, the deliverables described the mathematical operation used to assess the efficacy of the biomarker panel in appropriately determining the outcome variables, i.e. the risk of sepsis onset.

$$logit ( P ) = a + {bX}_{1} + {cX}_{2} + … + {nX}_{n} \qquad ( Equation 1 )$$

where logit( ) is the log odds function of a value, P that is the probability of successful determination of risk of sepsis onset. Here, P is determined by the area under the curve (AUC) of Receiver operating characteristic (ROC) curve. In the equation 1, a is the intercept of the equation, b through n are coefficient estimates of the independent variables, and X₁ through Xₙ are the expression values of the transcript 1 to transcript n, respectively. The fitting criteria of these probe combinations were measured by multiple R², adjusted R² and p values (Chi-square).

Table 1A provides information of 25 identified differentially expressed genes and probes used in detecting expression products of such genes, as follows:
1. Gene symbol: Gene symbols of the 25 genes identified, the sepsis biomarkers
2. corr.logfc: Correlation values between the gene expression data produced by targeted qPCR and high throughput microarray assays.
3. corr.logfc_p.value: The significance levels or p-values associated with the correlation values between the gene expression data produced by targeted qPCR and high throughput microarray assays.
4. average.logfc_array: Log₂(fold change) data produced by high throughput microarray assays.
5. average.logfc_qpcr: Log₂(fold change) data produced by targeted qPCR
6. Probe SEQ ID NOs: The sequences of the transcripts linked to the gene symbols are provided in the SEQ ID Listing submitted herewith.

Table 1B provides the full transcripts of the noted genes in Table 1A.

Table 2A describes the model delivered by RSBMR, and includes the names of the gene panels analyzed along with the appropriate intercepts and coefficients for Equation 1, as follows:
1. GeneName: List of gene symbols from the 25 gene set, which formed the panel
2. Intercept: Intercept of the equation as defined in Equation 1.
3. Gene1: The coefficient estimates of the Gene 1 of the panel as defined in Equation 1
4. Gene2: The coefficient estimates of the Gene 2 of the panel as defined in Equation 1 (if needed, otherwise noted as NA)
5. Gene3: The coefficient estimates of the Gene 3 of the panel as defined in Equation 1 (if needed, otherwise noted as NA)
6. Gene4: The coefficient estimates of the Gene 4 of the panel as defined in Equation 1 (if needed, otherwise noted as NA)
7. Gene5: The coefficient estimates of the Gene 5 of the panel as defined in Equation 1 (if needed, otherwise noted as NA)
8. Gene6: The coefficient estimates of the Gene 6 of the panel as defined in Equation 1 (if needed, otherwise noted as NA)

Table 2B provides values for the gene panels of Table 2A as follows:
1. GeneName: List of gene symbols from the 25 gene set, which formed the panel
2. P.Value: p-values showing the significance of fitting parameter
3. R.Squared: R² values showing the goodness of the fitting curve
4. Adjusted.R.Square: R² values showing the goodness of the fitting curve
5. Validation. Error: Error involved with the goodness of the fitting curve
6. AUC_Mean: Average AUC values of the ROC curves defined by all the random bins created from the cohorts
7. AUC_Median: Median AUC values of the ROC curves defined by all the random bins created from the cohorts
8. AUC_Min: Minimum AUC values of the ROC curves defined by all the random bins created from the cohorts
9. AUC_Max: Maximum AUC values of the ROC curves defined by all the random bins created from the cohorts

Table 2C provides values for the gene panels of Table 2A as follows:
1. Gene Name
2. Sensitivity_Mean: Average sensitivity score calculated from the ROC curves defined by all the random bins created from the cohorts
3. Sensitivity_Median: Median sensitivity score calculated from the ROC curves defined by all the random bins created from the cohorts
4. Sensitivity_Min: Minimum sensitivity score calculated from the ROC curves defined by all the random bins created from the cohorts
5. Sensitivity_Max: Maximum sensitivity score calculated from the ROC curves defined by all the random bins created from the cohorts
6. Specificity_Mean: Average specificity score calculated from the ROC curves defined by all the random bins created from the cohorts
7. Specificity_Median: Median specificity score calculated from the ROC curves defined by all the random bins created from the cohorts
8. Specificity_Min: Minimum specificity score calculated from the ROC curves defined by all the random bins created from the cohorts
9. Specificity_Max: Maximum specificity score calculated from the ROC curves defined by all the random bins created from the cohorts

Table 3A describes the model delivered by the k-fold algorithm, and includes the intercepts and coefficients for Equation 1 as follows. Explanation of the headers is as follows:
1. GeneName: List of gene symbols from the 25 gene set, which formed the panel
2. Intercept: Intercept of the equation as defined in Equation 1.
3. Gene1: The coefficient estimates of the Gene 1 of the panel as defined in Equation 1
4. Gene2: The coefficient estimates of the Gene 2 of the panel as defined in Equation 1 (if needed, otherwise noted as NA)
5. Gene3: The coefficient estimates of the Gene 3 of the panel as defined in Equation 1 (if needed, otherwise noted as NA)
6. Gene4: The coefficient estimates of the Gene 4 of the panel as defined in Equation 1 (if needed, otherwise noted as NA)
7. Gene5: The coefficient estimates of the Gene 5 of the panel as defined in Equation 1 (if needed, otherwise noted as NA)
8. Gene6: The coefficient estimates of the Gene 6 of the panel as defined in Equation 1 (if needed, otherwise noted as NA)

Table 3B provides the following values for the panels of Table 3A:
1. GeneName: List of gene symbols from the 25 gene set, which formed the panel
2. PanelSize: Number of genes included in the panel
3. P.Value: p-values showing the significance of fitting parameter
4. R.Squared: R² values showing the goodness of the fitting curve
5. Adjusted.R.Square: R² values showing the goodness of the fitting curve
6. Validation. Error: Error involved with the goodness of the fitting curve
7. AUC: AUC values of the ROC curves defined by cohort curated by k-fold algorithm
8. Sensitivity: Sensitivity score determined from the ROC curves defined by cohort curated by k-fold algorithm
9. Specificity: Specificity score determined from the ROC curves defined by cohort curated by k-fold algorithm

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

### REFERENCES

1 Kaukonen, K.-M., Bailey, M., Pilcher, D., Cooper, D. J. & Bellomo, R. Systemic inflammatory response syndrome criteria in defining severe sepsis. New England Journal of Medicine 372, 1629-1638 (2015).
2 Haydar, S., Spanier, M., Weems, P., Wood, S. & Strout, T. Comparison of QSOFA score and SIRS criteria as screening mechanisms for emergency department sepsis. The American journal of emergency medicine 35, 1730-1733 (2017).
3 Mann-Salinas, E. A. et al. Novel predictors of sepsis outperform the American Burn Association sepsis criteria in the burn intensive care unit patient. J Burn Care Res 34, 31-43, doi:10.1097/BCR.0b013e31826450b5 (2013).
4 McHugh, R. B. B. L. C. BIOMARKER IDENTIFICATION. USA patent (2019).
5 Greenhalgh, D. G. Sepsis in the burn patient: a different problem than sepsis in the general population. Burns Trauma 5, 23, doi:10.1186/s41038-017-0089-5 (2017).
6 Buehler, S. S. et al. Effectiveness of practices to increase timeliness of providing targeted therapy for inpatients with bloodstream infections: a laboratory medicine best practices systematic review and meta-analysis. Clinical microbiology reviews 29, 59-103 (2016).
7 Weinstein, M. P., Murphy, J. R., Reller, L. B. & Lichtenstein, K. A. The clinical significance of positive blood cultures: a comprehensive analysis of 500 episodes of bacteremia and fungemia in adults. II. Clinical observations, with special reference to factors influencing prognosis. Reviews of infectious diseases 5, 54-70 (1983).
8 Lee, C.-C., Chen, S.-Y., Chang, I.-J., Chen, S.-C. & Wu, S.-C. Comparison of clinical manifestations and outcome of community-acquired bloodstream infections among the oldest old, elderly, and adult patients. Medicine 86, 138-144 (2007).
9 Weinstein, M. P. et al. The clinical significance of positive blood cultures in the 1990s: a prospective comprehensive evaluation of the microbiology, epidemiology, and outcome of bacteremia and fungemia in adults. Clinical Infectious Diseases 24, 584-602 (1997).
10 Elixhauser, A., Friedman, B. & Stranges, E. Septicemia in US hospitals, 2009: statistical brief #122. (2006).
11 Novosad, S. A. Vital signs: epidemiology of sepsis: prevalence of health care factors and opportunities for prevention. MMWR. Morbidity and mortality weekly report 65 (2016).
12 Torio, C. & Andrews, R. National inpatient hospital costs: the most expensive conditions by payer, 2011: statistical brief #160. (2006).
13 Epstein, L. Varying estimates of sepsis mortality using death certificates and administrative codes—United States, 1999-2014. MMWR. Morbidity and mortality weekly report 65 (2016).
14 Klevens, R. M., Edwards, J. R., Gaynes, R. & System, N. N. I. S. The impact of antimicrobial-resistant, health care-associated infections on mortality in the United States. Clinical infectious diseases 47, 927-930 (2008).
15 Coburn, B., Morris, A. M., Tomlinson, G. & Detsky, A. S. Does this adult patient with suspected bacteremia require blood cultures? Jama 308, 502-511 (2012).
16 Singer, M. et al. The Third International Consensus Definitions for Sepsis and Septic Shock (Sepsis-3). JAMA 315, 801-810, doi:10.1001/jama.2016.0287 (2016).
17 Sinha, M. et al. Emerging Technologies for Molecular Diagnosis of Sepsis. Clin Microbiol Rev 31, doi:10.1128/CMR.00089-17 (2018).
18 Miller, R. R., 3rd et al. Validation of a Host Response Assay, SeptiCyte LAB, for Discriminating Sepsis from Systemic Inflammatory Response Syndrome in the ICU. Am J Respir Crit Care Med 198, 903-913, doi:10.1164/rccm.201712-2472OC (2018).
19 Nunez Lopez, O., Cambiaso-Daniel, J., Branski, L. K., Norbury, W. B. & Herndon, D. N. Predicting and managing sepsis in burn patients: current perspectives. Ther Clin Risk Manag 13, 1107-1117, doi:10.2147/TCRM.S119938 (2017).
20 Cabral, L., Afreixo, V., Santos, F., Almeida, L. & Paiva, J. A. Procalcitonin for the early diagnosis of sepsis in burn patients: A retrospective study. Burns 43, 1427-1434, doi:10.1016/j.burns.2017.03.026 (2017).
21 Sridharan, P. & Chamberlain, R. S. The efficacy of procalcitonin as a biomarker in the management of sepsis: slaying dragons or tilting at windmills? Surg Infect (Larchmt) 14, 489-511, doi:10.1089/sur.2012.028 (2013).
22 Shupp, J. W. et al. Military Supplement: Assessment of Coagulation Homeostasis in Blunt, Penetrating, and Thermal Trauma: Guidance for a Multi-Center Systems Biology Approach. Shock, doi:10.1097/SHK.0000000000001275 (2018).

Lengthy table referenced here
US20250188541A1-20250612-T00001
Please refer to the end of the specification for access instructions.

Lengthy table referenced here
US20250188541A1-20250612-T00002
Please refer to the end of the specification for access instructions.

Lengthy table referenced here
US20250188541A1-20250612-T00003
Please refer to the end of the specification for access instructions.

Lengthy table referenced here
US20250188541A1-20250612-T00004
Please refer to the end of the specification for access instructions.

Lengthy table referenced here
US20250188541A1-20250612-T00005
Please refer to the end of the specification for access instructions.

Lengthy table referenced here
US20250188541A1-20250612-T00006
Please refer to the end of the specification for access instructions.

LENGTHY TABLES
The patent application contains a lengthy table section. A copy of the table is available in electronic form from the USPTO web site (https://seqdata.uspto.gov/?pageRequest=docDetail&DocID=US20250188541A1). An electronic copy of the table will also be available from the USPTO upon request and payment of the fee set forth in 37 CFR 1.19(b)(3).

1. A method of identifying a burn subject at risk of developing sepsis, the method comprising,
a. measuring one or more biomarkers in a sample obtained from the burn subject to obtain one or more biomarker values, wherein the one or more biomarkers comprise an expression product, or a fragment or variant thereof, of a gene selected from ARG1A, ARG1B, ATG2A, BCL2A1, BMX, CD177, CEACAM4, CLEC4D, CLEC4D_A, HP, HPR, IL18R1, IL18RAP, MMP8, MS4A4A, PADI4, PFKFB2, PLAC8_A, RNASE2, SIGLEC5, STOM, TDRD9, VNN1, VNN1_2, or ZDHHC20;
b. determining that the burn subject is at risk of developing sepsis based on the biomarker values of the one or more biomarkers in the first sample; and
c. administering to the burn subject an effective amount of a sepsis therapy.

2. The method of claim 1, wherein the measuring one or more biomarkers in the sample comprises a molecular assessment.

3. The method of claim 1, wherein the molecular assessment comprises a nucleic acid sequencing assay, a next generation nucleic acid sequencing (NGS) assay, a Sanger sequencing assay, a PCR assay, a quantitative PCR (qPCR) assay, a reverse transcription PCR (RT-PCR) assay, a miRNA assay, a microarray assay, a Northern blot assay, a Southern blot assay, a luciferase assay, a fluorescence immunoassay, a radio immunoassay, an enzyme-linked immunosorbent assay (ELISA), a flow cytometry assay, a mass spectrometry (MS) assay, a Selected Reaction Monitoring (SRM-MS) assay, a Sequential Windowed data independent Acquisition of the Total High resolution Mass Spectroscopy (SWATH-MS) assay, a Western blot assay, a genome wide methylation assay, a targeted methylation assay, a bisulfite methylation sequencing assay, a restriction enzyme methylation sequencing assay, a high performance liquid chromatography (HPLC) assay, an ultrahigh performance liquid chromatography (UHPLC) assay, a mass spectrometry (MS) assay, an ultrahigh performance liquid chromatography/tandem mass spectrometry (UHPLC/MS/MS2), or a gas chromatography/mass spectrometry (GC/MS) assay.

4. The method of claim 1, wherein the sample comprises a whole blood sample, a plasma sample, a serum sample, a huffy coat sample, a peripheral blood mononuclear cell sample, a CSF sample, a urine sample, a saliva sample, a sweat sample, a prefrontal cortex tissue sample, a hippocampus tissue sample, or an ipsilateral cortex tissue sample.

5. The method of claim 1, wherein the sepsis therapy comprises an antibiotic, intravenous hydration, transfusion of blood products, a vasopressor, ventilator assistance, a non-steroidal anti-inflammatory agent, or an anti-pyretic agent.

6. The method of claim 1, wherein measuring occurs within 12-36 hours, or within 24 hours, of the burn subject being admitted to an intensive care unit.

7. The method of claim 1, wherein the determining step comprises: multiplying said one or more biomarker values by one or more predetermined coefficients for the one or more biomarkers to obtain one or more products and adding the one or more products to obtain a total risk score that corresponds to a probability of developing sepsis.

8. The method of claim 7, wherein measuring one or more biomarkers comprises measuring an amount of expression products of a panel of 2-6 genes; wherein the panel is set forth in Tables 2A and 3A.

9. (canceled)

10. The method of claim 8, wherein the panel is ARG1B, BMX, HP, IL18R1, MS4A4A, and ZDHHC20.

11. The method of claim 7, wherein the one or more predetermined coefficients are set forth in Tables 2A and 2B.

12. The method of claim 7, wherein the one or more biomarker values comprises transcript values of the one or more genes, and wherein the determining step comprises obtaining a total risk score according to the following equation:
$$logit ( P ) = a + {bX}_{1} + {cX}_{2} + … + {nX}_{n} \qquad ( Equation 1 )$$
where logit( ) is the logistic regression P value, which is the probability of successful determination of risk of sepsis onset, a is the intercept of the equation as is set forth in Tables 2A and 3A, b through n are coefficient estimates of the independent variables as set forth in Tables 2A and 3A, and X₁ through Xₙ are the expression values of the transcript 1 to transcript n, respectively.

13. The method of claim 12, wherein logit(P) empirically determine the area under the curve (AUC) of Receiver operating characteristic (ROC) curve.

14. The method of claim 7, further comprising communicating said probability to a health care provider.

15. A method, in a computer-implemented system comprising at least one processor and at least one memory, the at least one memory comprising instructions executed by the at least one processor to cause the at least one processor to implement a predictive model to predict an increased risk of a burn subject patient to develop sepsis, the method comprising:
a) obtaining measured values of a panel of biomarkers in a sample from the burn subject, wherein a value of a biomarker corresponds to a level of the biomarker in the sample;
b) classifying the burn subject into a risk category of developing sepsis using a predictive model, wherein the predictive model is generated by a machine learning system using first training data that comprises values of a panel of at least two biomarkers; and,
wherein the predictive model classifies the patient in an increased risk category using input variables of the measured values of a panel of biomarkers from the burn subject when an output of the predictive model is above a threshold; and,
c) providing a notification to a user that the burn subject is classified in the increased risk category.

16. The method of claim 15, wherein the first classifier model has a performance of a Receiver Operator Characteristic (ROC) curve with a sensitivity value of at least 0.8 and a specificity value of at least 0.65.

17. The method of claim 15, wherein the input variables comprise measured values of expression products of a panel of at least 2-6 biomarker genes; wherein, optionally, the panel of biomarker genes is selected from ARG1A, ARG1B, ATG2A, BCL2A1, BMX, CD177, CEACAM4, CLEC4D, CLEC4D_A, HP, HPR, IL18R1, IL18RAP, MMP8, MS4A4A, PADI4, PFKFB2, PLAC8 A, RNASE2, SIGLEC5, STOM, TDRD9, VNN1, VNN1_2, or ZDHHC20.

18. (canceled)

19. The method of claim 17, wherein the panel of biomarker genes is ARG1B, BMX, HP, IL18R1, MS4A4A, and ZDHHC20.

20. (canceled)

21. (canceled)

22. A detecting kit comprising primers and probes for detecting 2 or more expression products of 2 or more genes selected from ARG1A, ARG1B, ATG2A, BCL2A1, BMX, CD177, CEACAM4, CLEC4D, CLEC4D_A, HP, HPR, IL18R1, IL18RAP, MMP8, MS4A4A, PADI4, PFKFB2, PLAC8_A, RNASE2, SIGLEC5, STOM, TDRD9, VNN1, VNN1_2, or ZDHHC20.

23. The detecting kit of claim 22, wherein the 2 or more genes are ARG1B, BMX, HP, IL18R1, MS4A4A, and ZDHHC20 or wherein the probes comprise two or more probes selected from SEQ ID NOs 1-25, or both.

24. (canceled)

25. The detecting kit of any of claim 22, the kit comprising a set of probes and a set of oligonucleotide primer pairs, wherein each probe of the set specifically binds to one distinct biomarker, and each set of oligonucleotide primer pairs specifically amplifies a distinct biomarker, wherein at least one member of each set of probes or at least one member of each set of oligonucleotide primer pairs binds to a biomarker ARG1A, ARG1B, ATG2A, BCL2A1, BMX, CD177, CEACAM4, CLEC4D, CLEC4D_A, HP, HPR, IL18R1, IL18RAP, MMP8, MS4A4A, PADI4, PFKFB2, PLAC8_A, RNASE2, SIGLEC5, STOM, TDRD9, VNN1, VNN1_2, or ZDHHC20, and optionally, wherein said probes or set of oligonucleotide primer pairs are provided on a solid substrate; and optionally a control or reference sample.

26. (canceled)

27. (canceled)

28. (canceled)
